(19) United States
(12) Patent Application Publication
(10) Pub. No.: US 2021/0086778 A1
(43) Pub. Date: Mar. 25, 2021

(54) IN-VEHICLE EMERGENCY DETECTION AND RESPONSE HANDLING

(71) Applicant: OLA ELECTRIC MOBILITY PRIVATE LIMITED, Bengaluru (IN)

(72) Inventors: Parth Suthar, Bangalore (IN); Sudhir Singh Mor, Ahmedabad (IN); Shreeyash Salunke, Pune (IN); Arijit Dey, Agartala (IN); Mihul Prakash, Kalyanpur (IN); Arjun S, Bengaluru (IN); Manu Chaudhary, Ghaziabad (IN); Poorva Mankad, Bengaluru (IN); Ravi Shankar Singh Ahirwar, Bhopal (IN)

(73) Assignee: OLA ELECTRIC MOBILITY PRIVATE LIMITED, Bengaluru (IN)

(21) Appl. No.: 17/027,163

(22) Filed: Sep. 21, 2020

(30) Foreign Application Priority Data
Sep. 23, 2019 (IN) 201941029815

(51) Int. Cl. B60W 40/08; B60W 60/00; B60Q 1/52; B60Q 1/50; G10L 15/22; G06F 3/01; A61B 5/00; H04W 4/90

(52) U.S. Cl. CPC B60W 40/08; B60W 60/0016; B60Q 1/52; B60Q 1/503; G10L 2015/228; G06F 3/017; A61B 5/6893; H04W 4/90; B60W 2540/221; G10L 15/22

(57) ABSTRACT

A method for emergency response handling includes receiving an emergency signal from at least one of a vehicle or a user device of an occupant of the vehicle. Sensor data from one or more sensors in the vehicle or the user device is collected. An in-vehicle emergency is detected based on the sensor data and a severity level and a context associated with the in-vehicle emergency are determined. From a plurality of responses, one or more responses are selected, based on the determined severity level and context, for handling the in-vehicle emergency. Response actions are executed based on the selected responses. Execution of the response actions includes communicating, an instruction to at least one of the user device, an emergency contact, a response team, and user devices associated with users present within a first distance from the vehicle.

### CROSS-RELATED APPLICATIONS

This application claims priority of Indian Provisional Application No. 201941029815, filed Sep. 23, 2019, the contents of which are incorporated herein by reference.

### FIELD

Various embodiments of the disclosure relate generally to emergency assistance. More specifically, various embodiments of the disclosure relate to methods and systems for in-vehicle emergency detection and response handling.

### BACKGROUND

In-vehicle emergencies may be caused due to accidental damages, medical concerns, criminal activities, or the like. An in-vehicle emergency may put a vehicle as well as an occupant of the vehicle in harm's way. Further, assistance required for resolving different emergencies may vary based on a time, a place, and a cause associated with each emergency.

A known solution for providing assistance for handling in-vehicle emergencies includes installation of emergency assistance devices within vehicles. An emergency assistance device reports the emergency based on information provided by an occupant of a vehicle. However, such emergency assistance devices face many performance and technical challenges. For example, the emergency assistance device requires continuous and uninterrupted connection to a network for reporting emergencies. Thus, when the network is unavailable, the emergency assistance device is unable to report any emergency. Further, the emergency assistance device reports the emergency based on an input provided by the occupant. However, in case of an accident or a medical emergency, the occupant may be unable to provide the input, which may lead to an undesirable and critical situation. In addition, such emergency assistant devices are not efficient in terms of determining severity and authenticity of the emergency, thus resulting in frequent false negative and false positive incidents. Further, a single emergency assistance device may not be suitable for addressing different types of emergencies. Therefore, relying on such emergency assistance devices may not be an effective solution for handling different types of emergencies.

In light of the foregoing, there exists a need for a technical and reliable solution that overcomes the above-mentioned problems and allows for accurate and efficient handling of in-vehicle emergencies.

### SUMMARY

Methods for in-vehicle emergency detection and response handling are provided substantially as shown in, and described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates a system environment for in-vehicle emergency detection and response handling, in accordance with an exemplary embodiment of the disclosure;

FIG. 2 is a block diagram that illustrates an application server of FIG. 1, in accordance with an exemplary embodiment of the disclosure;

FIG. 3 is a schematic diagram that illustrates an exemplary scenario of a top view of the vehicle of FIG. 1, in accordance with an exemplary embodiment of the disclosure;

FIG. 4 is a schematic diagram that illustrates a user interface rendered by the application server of FIG. 1 on a user device, in accordance with an exemplary embodiment of the disclosure;

FIG. 5 is a block diagram that illustrates an exemplary environment for in-vehicle emergency detection and response handling, in accordance with an exemplary embodiment of the disclosure;

FIG. 6 is a block diagram that illustrates another exemplary environment for in-vehicle emergency detection and response handling, in accordance with an exemplary embodiment of the disclosure;

FIG. 7 is a block diagram that illustrates another exemplary environment for in-vehicle emergency detection and response handling, in accordance with an exemplary embodiment of the disclosure;

FIG. 8 is a block diagram that illustrates a system architecture of a computer system for in-vehicle emergency detection and response handling, in accordance with an exemplary embodiment of the disclosure;

FIGS. 9A and 9B, collectively illustrate a flowchart of a method in-vehicle emergency detection and response handling, in accordance with an exemplary embodiment of the disclosure; and

FIGS. 10A, 10B, and 10C, collectively illustrate an exemplary flowchart of a method for in-vehicle emergency detection and response handling, in accordance with an exemplary embodiment of the disclosure.

### DETAILED DESCRIPTION

Certain embodiments of the disclosure may be found in disclosed systems and methods for in-vehicle emergency detection and response handling. Exemplary aspects of the disclosure provide methods for emergency detection and response handling. The methods include various operations that are executed by a server (for example, an application server) to handle emergency responses for resolving in-vehicle emergencies. In an embodiment, the server is configured to receive an emergency signal from at least one of a vehicle or a user device of an occupant of the vehicle. The server is further configured to collect sensor data, based on the emergency signal, from one or more sensors in the vehicle or the user device. The server is further configured to detect the in-vehicle emergency based on the sensor data. The in-vehicle emergency corresponds to at least one of an emergency associated with the occupant and an emergency associated with the vehicle. The server is configured to determine a severity level and a context associated with the detected in-vehicle emergency based on the sensor data. The server is further configured to select from a plurality of responses, one or more responses to handle the detected in-vehicle emergency. The one or more responses are selected based on the determined severity level and the determined context. The server is further configured to execute one or more response actions based on the selected one or more responses. The server is configured to execute the one or more response actions by communicating an instruction to at least one of the user device, an emergency contact of the occupant, a response team associated with the in-vehicle emergency, and one or more user devices associated with one or more users present within a first distance from the vehicle when the in-vehicle emergency is detected.

In one embodiment, the server is configured to receive the emergency signal based on an input received via one of the one or more sensors, a mobile application hosted on the user device, and an emergency input interface of the vehicle.

In another embodiment, the server is configured to store a medical profile of the occupant in a memory of the user device. The emergency signal is received based on a deviation of a current health state of the occupant from the stored medical profile.

In one embodiment, the one or more sensors include at least one of a global positioning system (GPS) sensor, an impact sensor, a temperature sensor, a speed sensor, a smoke sensor, a proximity sensor, a pressure sensor, an image sensor, and a microphone.

In another embodiment, the server is further configured to cause an external vehicular display of the vehicle to display textual and graphical content indicating the detected in-vehicle emergency. In another embodiment, the server is further configured to communicate a message or a pre-recorded voice message to the emergency contact of the occupant. In another embodiment, the server is configured to display an emergency assistance message on the user device of the occupant.

In one embodiment, the server is configured to provide a navigation assistance to the occupant of the vehicle via the user device or a vehicle device of the vehicle. In another embodiment, the server is configured to control remotely, a central lock of the vehicle for locking or unlocking. In another embodiment, the server is configured to control remotely, a driving speed of the vehicle to halt the vehicle. The server is further configured to initiate a troubleshooting of the vehicle.

In one embodiment, the server is configured to activate the one or more sensors, based on the emergency signal, for the collection of the sensor data. In one embodiment, the sensor data includes at least one of image data, speech data of the occupant, temperature data, impact magnitude data, pressure magnitude data, speedometer data, location data, and current health state data. The current health state data includes blood pressure magnitude data of the occupant, pulse-rate data of the occupant, oxygen level data of the occupant, and state of consciousness of the occupant.

In another embodiment, the server is configured to store in a memory of one of the user device and a vehicle device of the vehicle, a plurality of emergency keywords indicating a plurality of in-vehicle emergencies. The emergency signal is received based on an utterance of one or more emergency keywords from the plurality of emergency keywords by the occupant.

In another embodiment, the server is configured to store, in the memory of one of the user device and the vehicle device of the vehicle, a plurality of gestures indicating a plurality of in-vehicle emergencies. The emergency signal is received based on an action of the occupant that results in one or more gestures of the plurality of gestures.

The methods and systems of the disclosure provide a solution for emergency response handling for the in-vehicle emergency. The methods and systems significantly improve efficiency and accuracy of in-vehicle emergency assistance. Beneficially, the methods and systems substantially reduce human intervention for initiating the emergency assistance to resolve the in-vehicle emergency. Further, the disclosed methods and systems initiate emergency assistance not only based on received inputs but also by detecting the in-vehicle emergency based on the sensor data collected from the one or more sensors in the vehicle. Therefore, the disclosed methods and systems significantly reduces a likelihood of false-positive or false-negative incidents. Further, the disclosed methods and systems ensure that the in-vehicle emergency is handled in a timely and optimal manner.

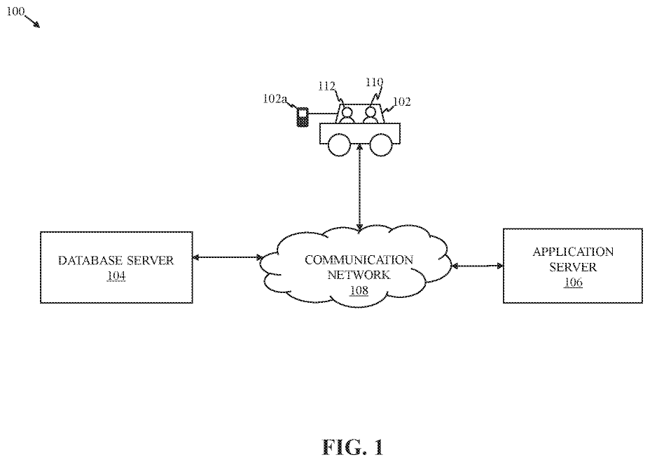

FIG. 1 is a block diagram that illustrates a system environment 100 for in-vehicle emergency detection and response handling, in accordance with an exemplary embodiment of the disclosure. The environment 100 includes a vehicle 102, a database server 104, an application server 106, and a communication network 108. The environment 100 further includes a user device 102a. In an embodiment, the vehicle 102, the database server 104, and the application server 106 may communicate with each other by way of the communication network 108. Examples of the communication network 108 may include, but are not limited to, a wireless fidelity (Wi-Fi) network, a light fidelity (Li-Fi) network, a local area network (LAN), a wide area network (WAN), a metropolitan area network (MAN), a satellite network, the Internet, a fiber optic network, a coaxial cable network, an infrared (IR) network, a radio frequency (RF) network, and a combination thereof. Various entities (such as the vehicle 102, the user device 102a, the database server 104, and the application server 106) in the system environment 100 may be coupled to the communication network 108 in accordance with various wired and wireless communication protocols, such as Transmission Control Protocol and Internet Protocol (TCP/IP), User Datagram Protocol (UDP), Long Term Evolution (LTE) communication protocols, or any combination thereof. In one example, the vehicle 102 may be communicatively coupled to the communication network 108 via one of a telematics device, an on-board diagnostics device (OBD), or the user device 102a.

The vehicle 102 is a mode of transport that is used by a user to commute from one location to another location. The user may correspond to a driver 110 or a passenger 112. In one example, the driver 110 driving the vehicle 102 may be the sole occupant of the vehicle 102. In another example, the driver 110 and the passenger 112 may be travelling together in the vehicle 102. In an exemplary scenario, the driver 110 may be associated with a vehicle service provider (e.g., a ride-hailing service provider such as OLA) that offers on-demand vehicle services to passengers, such as the passenger 112. Examples of the vehicle 102 may include, but are not limited to, an automobile, a car, an auto-rickshaw, a bike, or the like. For the sake of brevity, it is assumed that the driver 110 and the passenger 112 are the two occupants of the vehicle 102. Hereinafter, the term “the driver 110” is referred to as “occupant 110” and the term “the passenger 112” is referred to as “occupant 112” throughout the disclosure.

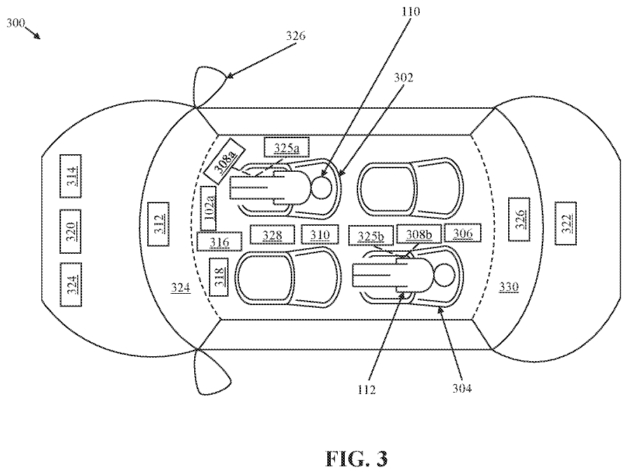

In an exemplary embodiment, the vehicle 102 includes a plurality of sensors (as shown in FIG. 3) installed therein. The plurality of sensors may include a global positioning system (GPS) sensor, an impact sensor, a temperature sensor, a speed sensor, a smoke sensor, a proximity sensor, an alcohol sensor, and a pressure sensor (as shown in FIG. 3). The plurality of sensors may be configured to detect and monitor health conditions of the vehicle 102. The plurality of sensors further include an image sensor (as shown in FIG. 3) for capturing or monitoring in-vehicle objects, activities, or incidents. The plurality of sensors further include a microphone (as shown in FIG. 3) for capturing and monitoring in-vehicle audio including one or more emergency keywords uttered by the occupants 110 and 112. The plurality of sensors further include sensors that detect and monitor health conditions of the occupants 110 and 112, in real-time. Such sensors may include, but are not limited to, a heart rate sensor, a blood pressure sensor, a body sensor, and an InfraRed (IR) sensor. The heart rate sensor and the blood pressure sensor may measure and monitor the heart rate and the blood pressure of the occupants 110 and 112. The body sensor may measure and monitor various vital signs of the occupants 110 and 112. The IR sensor may measure and monitor body temperatures of the occupants 110 and 112.

In one embodiment, the vehicle 102 may further include one or more fitness accessories to be used by the occupants 110 and 112. Further, the vehicle 102 may include an alcohol detector for detecting an alcohol level of the occupants 110 and 112. In another embodiment, the fitness accessories may be owned by the occupants 110 and 112. The fitness accessories may be configured to monitor the health conditions of the occupants 110 and 112. Each fitness accessory may include one or more sensors such as a blood pressure sensor, a pulse rate sensor, a movement sensor, an alcohol sensor, or the like. Examples of the fitness accessories may include fitness watches (not shown), fitness bands (not shown), or the like, that may be worn by the occupants 110 and 112. The fitness accessories may generate current health state data based on a current health state of the corresponding occupants 110 and 112. For example, the current health state data of the occupant 110 may include blood pressure magnitude data of the occupant 110, pulse-rate data of the occupant 110, oxygen level data of the occupant 110, state of consciousness of the occupant 110, state of dizziness of the occupant 110, and an alcohol level of the occupant 110.

The vehicle 102 further includes one or more emergency input interfaces such as emergency input buttons (as shown in FIG. 3) for receiving manual inputs from the occupants 110 and 112. The plurality of sensors may generate sensor data including image data, speech data of the occupants 110 and 112, temperature data, impact magnitude data, pressure magnitude data, speedometer data, and location data. In one embodiment, the sensor data may further include the current health state data.

In one embodiment, the user device 102a may be a vehicle head unit associated with the vehicle 102. In another embodiment, the user device 102a may be associated with one of the occupants 110 and 112. Examples of the user device 102a may include a cell-phone, a laptop, a tablet, a phablet, an in-vehicle multi-tainment device, or the like. The user device 102a may be configured to run a mobile application. In one embodiment, the mobile application may be hosted by the application server 106. The mobile application may be used by the occupant 110 or the occupant 112 to report an emergency incident.

In one embodiment, the user device 102a may be configured to synchronize with the plurality of sensors and a fitness accessory by way of the mobile application. In one embodiment, the mobile application running on the user device 102a may automatically initiate an emergency signal based on the sensor data received from at least one of the plurality of sensors or the fitness accessory. In another embodiment, the mobile application may initiate the emergency signal based on an input provided by at least one of the occupants 110 and 112.

The database server 104 may include suitable logic, circuitry, interfaces, and/or code, executable by the circuitry, that may be configured to perform one or more operations for collecting, storing, processing, and transmitting queries, data, or content for providing emergency assistance for resolving the in-vehicle emergency. In an embodiment, the database server 104 may store information required for emergency response handling by the application server 106. The database server 104 may be communicatively coupled to the application server 106 by way of the communication network 108. In one embodiment, the database server 104 may be communicatively coupled to the user device 102a.

The application server 106 is a system that includes suitable logic, circuitry, interfaces, and/or code, executable by the circuitry, that may be configured to perform one or more operations for providing emergency assistance to resolve in-vehicle emergencies. The application server 106 may be configured to communicate with the vehicle 102, the user device 102a, and the database server 104. Various operations of the application server 106 may be dedicated to execution of procedures, such as, but not limited to, programs, routines, or scripts stored in its memory for supporting the one or more operations for emergency response handling. The application server 106 may be realized through various web-based technologies, such as, but not limited to, a Java web-framework, a .NET framework, a PHP framework, or any other web application framework. Examples of the application server 106 may include, but are not limited to, a personal computer, a laptop, or a network of computer systems.

An in-vehicle emergency may refer to an undesirable situation or incident associated with the vehicle 102 and/or any of the occupants 110 and 112 of the vehicle 102. The in-vehicle emergency associated with the vehicle 102 may be an accident or an unlawful incident such as a burglary, a theft, a counterfeit, or the like. The in-vehicle emergency associated with the any of the occupants 110 and 112 may be one of a medical emergency, an accident, an unlawful incident, or a combination of these. Examples of the medical emergency may include, but are not limited to, a heart stroke, an asthma attack, a drop in sugar level, or the like. Examples of the accident may include, but are not limited to, a collision of the vehicle 102 with another vehicle or an obstacle (such as a tree, a wall, a lane divider, and the like), a failure of brakes of the vehicle 102, a fire in engine of the vehicle 102, and/or the like. Examples of the unlawful incident may include, but are not limited to, a burglary on knife-point, an abduction, a physical assault, and/or a brawl leading to a physical altercation between the occupants 110 and 112, the occupant 112 and another ride sharing passenger of the vehicle 102, or the like.

The application server 106 may be configured to store a plurality of emergency keywords and a plurality of gestures corresponding to a plurality of in-vehicle emergencies in the memory thereof. Further, the application server 106 may also be configured to store emergency contacts of the occupants 110 and 112 in the memory. In one example, the emergency contacts of the occupants 110 and 112 may be provided by the occupants 110 and 112 before the start of the ride in the vehicle 102. In another embodiment, the application server 106 may be configured to access contact lists (e.g., a phonebook) available on communication devices of the of the occupants 110 and 112 upon their consent, and retrieve the emergency contacts of the occupants 110 and 112. In an embodiment, the application server 106 may be configured to locally store the plurality of emergency keywords and the plurality of gestures in a memory of the user device 102a.

The application server 106 may be configured to receive an emergency signal based on an input received via the plurality of sensors, the mobile application running on the user device 102a or any other device in the vehicle 102, and the emergency input interface of the vehicle 102. In one example, the emergency signal may be generated when one of the occupants 110 and 112 presses a physical input button in the vehicle 102 or a touch-based input button provided on the user device 102a via the mobile application, to raise a safety concern or report an in-vehicle emergency. In another example, the emergency signal may be generated based on sensor data of an impact sensor installed in the vehicle 102. In another example, the emergency signal may be generated based on in-vehicle speech data, i.e., one or more emergency keywords uttered by any of the occupants 110 and 112. In another example, the emergency signal may be generated when one or more emergency keywords are uttered by any of the occupants 110 and 112 in a specific pitch that is greater than a threshold value. In another example, the emergency signal may be generated when the one or more emergency keywords are uttered by any of the occupants 110 and 112 for a specific number of times. In another example, the emergency signal is generated based on an action of the occupants 110 and 112 that results in a gesture from the plurality of gestures indicating the plurality of in-vehicle emergencies.

In an embodiment, the application server 106 may be further configured to receive, from the vehicle 102, or the user device 102a, a mode of generation (i.e., a mode of trigger) of the emergency signal. The mode of generation of the emergency signal may refer to a source of the emergency signal. The various modes of the emergency signal may be the mobile application, one or more sensors of the plurality of sensors, and the emergency input interface installed in the vehicle 102.

Upon reception of the emergency signal, the application server 106 may be configured to activate the plurality of sensors in the vehicle 102. The application server 106 may activate the plurality of sensors via one of the telematics device of the vehicle 102, the user device 102a, the OBD device of the vehicle 102, or the like. In an embodiment, the application server 106 may directly control the plurality of sensors. The application server 106 may activate the plurality of sensors to confirm validity of the emergency signal. The application server 106 may be further configured to collect real-time or near real-time sensor data from the activated plurality of sensors. The application server 106 may be configured to compare the received sensor data with normal sensor data stored in the database server 104 or the memory of the application server 106. The normal sensor data may include sensor data that indicates an absence of any in-vehicle emergency. The application server 106 may validate the emergency signal and detect the occurrence of the in-vehicle emergency based on a deviation in the collected sensor data from the stored normal sensor data. Beneficially, validating the emergency signal eliminates a probability of a false positive in-vehicle emergency. In one embodiment, the application server 106 may be configured to validate the emergency signal and detect the in-vehicle emergency based on the sensor data being greater than or equal to a first threshold value. In another embodiment, the application server 106 may be configured to validate the emergency signal and detect the in-vehicle emergency based on a comparison of the sensor data with historical sensor data that was collected to validate a historical in-vehicle emergency. An unsuccessful validation of the sensor data indicates that the received emergency signal is a false positive. A successful validation of the sensor data ensures that the emergency signal indeed corresponds to an in-vehicle emergency.

In an embodiment, the application server 106 may be configured to determine a validation score associated with the collected sensor data. The validation score may be determined by analyzing the collected sensor data based on one or more machine learning techniques known in the art. The validation score may be indicative of a likelihood or a probability of the emergency signal to be associated with a valid in-vehicle emergency.

In an embodiment, when the mode of trigger of the emergency signal may be a microphone, the mobile application may be configured to communicate recorded in-vehicle speech data to the application server 106. The application server 106 may be configured to apply one or more natural language processing (NLP) techniques to analyze the speech data. The application server 106 may analyze the speech data to determine a context of the speech data, a distress level of the speech data, a tonality of the speech data, or the like. The application server 106 may determine the context of the speech data by applying the NLP techniques to understand a meaning and an intent associated with the speech data. The application server 106, based on the context of the speech data, the distress level of the speech data, and the tonality of the speech data, may determine the validation score for the occurrence of the in-vehicle emergency. In an embodiment, the application server 106 may determine the validation score by using one or more machine learning models, trained based on data associated with historical emergency assistances.

In an exemplary scenario, the speech data may include “HELP, THE DOOR IS CRUSHING MY FINGERS. THANKS”. The application server 106, based on the analysis of the speech data, may determine that although one of the occupants 110 and 112 has uttered the emergency keywords “HELP” and “CRUSHING” that resulted in the generation of the emergency signal, a context of the speech data, a tonality of utterance, and a distress level in the utterance do not correspond to an in-vehicle emergency. Thus, the validation score in such a scenario is very low. In another exemplary scenario, the speech data may include “OH GOD, PLEASE NO, HELP! HELP! NO”. The application server 106, based on the analysis of the speech data, may determine that the context of the speech data may correspond to an attack or an assault. The application server 106 may further determine that the distress level in the speech data may be high. Further, the application server 106 may determine, based on the tonality of the speech data, that the speech data was uttered while shouting and trembling. Therefore, in this scenario, the application server 106 may determine the validation score to be high based on the distress level and the context of the speech data.

The application server 106 may be further configured to compare the determined validation score with a threshold score and when the determined validation score is greater than the threshold score, the application server 106 validates the emergency signal and detects the in-vehicle emergency. However, if the determined validation score is less than the threshold score, the application server 106 declares the emergency signal to be invalid or a false negative. The threshold score may represent a borderline between actual in-vehicle emergencies and false in-vehicle emergencies. The threshold score may be determined by the application server 106 based on analysis of historical in-vehicle emergencies and false in-vehicle emergencies.

In an embodiment, the application server 106 may be configured to store details (such as the mode of trigger of the emergency signal, the speech data, the image data, other sensor data, and/or the like) associated with historical in-vehicle emergencies in the memory thereof. The application server 106 may apply one or more supervised learning techniques to use the stored details for efficient handling of future in-vehicle emergencies.

In an embodiment, upon detecting an in-vehicle emergency that is different from the historical in-vehicle emergencies, the application server 106 may be configured to enable the occupants 110 and 112 to provide a feedback, via the user device 102a or any other device, regarding the in-vehicle emergency and the emergency assistance. The feedback may be obtained in one of a covert mode and an overt mode. The mode of obtaining the feedback may be determined by the application server 106 based on a type or category of the detected in-vehicle emergency. When the in-vehicle emergency corresponds to an unlawful incident, the feedback may be obtained in the covert mode. For example, in case of an abduction of the occupant 110, the application server 106 may be configured to initiate an interactive voice response (IVR) call on the user device 102a for obtaining the feedback from the occupant 110. The IVR call may only include “Yes” or “NO” questions to which the occupant 110 may reply by pressing relevant buttons of the user device 102a or by uttering “YES” or “NO”. In another example, the occupant 110, upon receiving the IVR call, may provide feedback by operating different devices (for example, a smart fare device) installed in the vehicle 102. Beneficially, the covert mode of obtaining the feedback allows the initiation of the emergency assistance to be imperceptible to others such as other occupants, a third party, and the like. When the in-vehicle emergency corresponds to a medical emergency, the feedback may be obtained in the overt mode. For example, in case of an accident of the vehicle 102, the application server 106 may be configured to initiate a call on the user device 102a and may prompt the occupant 110 to describe the emergency situation and a type of emergency assistance required for resolving the emergency. It will be apparent to a person of ordinary skill in the art that the abovementioned overt and covert modes of obtaining feedback are exemplary and do not limit the scope of the disclosure in any manner. The application server 106 may implement one or more other overt and covert modes for obtaining feedback without deviating from the scope of the disclosure.

The application server 106 may be further configured to determine, upon detection of the in-vehicle emergency, a severity level and a context of the detected in-vehicle emergency. The application server 106 may be configured to determine the severity level and the context of the detected in-vehicle emergency based on the collected sensor data. The severity level may refer to a criticality of an emergency situation (i.e., the in-vehicle emergency) associated with the vehicle 102 or the occupants 110 or 112. A high severity level may indicate a critical situation of the detected in-vehicle emergency. A high severity level may further indicate that immediate assistance is required for resolving the detected in-vehicle emergency. In an embodiment, the application server 106 may prioritize the detected in-vehicle emergency with high severity level over other non-severe in-vehicle emergencies. The context of the detected in-vehicle emergency may be determined based on one or more parameters including first physical factors associated with the vehicle 102, second physical factors associated with the occupants 110 and 112, medical factors associated with the occupants 110 and 112, infrastructure availability factors around a current location of the vehicle 102, environmental factors associated with a geographical region associated with the current location of the vehicle 102, and/or the like. In one embodiment, the context of the detected in-vehicle emergency may be determined based on the image data and the speech data collected by the application server 106. The context of the detected in-vehicle emergency may be further indicative of a type of assistance (i.e., infrastructure, response teams, and the like) required for handling the detected in-vehicle emergency.

In one embodiment, the application server 106 may analyze the speech data of each occupant 110 and 112 to determine the severity level and the context of the in-vehicle emergency. In an embodiment, the application server 106 may determine the severity level and the context of the detected in-vehicle emergency based on a distress level associated with the speech data, a pitch of words uttered by each occupant 110 or 112, a count of emergency keywords uttered by each occupant 110 or 112, a tonality of the speech data, a context of the speech data, and/or the like. For example, a pitch higher than a first pitch value may indicate a high severity level of the in-vehicle emergency and a pitch lower than the first pitch value may indicate non-severity of the in-vehicle emergency. In another embodiment, the application server 106 may determine the severity level of the in-vehicle emergency based on a count of emergency keywords uttered by the occupant 110 or 112. A count of emergency keywords uttered being higher than a keyword count threshold (for example, 5 words, 6 words, 7 words, and so forth) may indicate the high severity level of the detected in-vehicle emergency. A count of emergency keywords uttered being lower than the keyword count threshold may indicate low severity level of the detected in-vehicle emergency. In a first exemplary scenario, the occupant 110 may utter “HELP”. In a second exemplary scenario, the occupant 110 may utter “PLEASE HELP! I CAN'T BREATHE”. The application server 106, based on the count of emergency keywords, may determine that the severity level in the first exemplary scenario may be lower than the severity level in the second exemplary scenario. In another embodiment, the application server 106 may determine the severity level based on a first category and a second category of emergency keywords. The first category may include emergency keywords indicative of high severity level and the second category may include emergency keywords indicative of low severity level. In a third exemplary scenario, the occupant 110 may utter words “BRAKES FAIL”. In a fourth exemplary scenario, the occupant 110 may utter a word “ANXIOUS”. The words “brakes” and “fail” may belong to the first category and the word “anxious” may belong to the second category. Therefore, the application server 106 may determine that the detected in-vehicle emergency in the third exemplary scenario has a high severity level and the detected in-vehicle emergency in the fourth exemplary scenario has a low severity level. In one embodiment, the application server 106 may be configured to determine the severity level of the detected in-vehicle emergency based on an image of the occupant 110 or 112. In a fifth exemplary scenario, the image may show that the occupant 110 is alone and has his eyes closed. In a sixth exemplary scenario, the image may show that the occupant 110 has his eyes opened and is communicating with the other occupant 112. Therefore, the application server 106 may determine that the severity level of the detected in-vehicle emergency in the fifth exemplary scenario is higher than the severity level of the detected in-vehicle emergency in the sixth exemplary scenario.

In another embodiment, the application server 106 may determine the severity level of the detected in-vehicle emergency based on a magnitude of temperature indicated by sensor data received from the temperature sensor. A temperature higher than a temperature threshold may be indicative of high severity level of the detected in-vehicle emergency and a temperature lower than the temperature threshold may be indicative of low severity level of the detected in-vehicle emergency. In another embodiment, the application server 106 may determine the severity level of the detected in-vehicle emergency based on a magnitude of shock or vibration indicated by sensor data received from the impact sensor. A shock or vibration higher than a vibration threshold may be indicative of high severity level of the detected in-vehicle emergency and a vibration lower than the vibration threshold may be indicative of low severity level of the detected in-vehicle emergency.

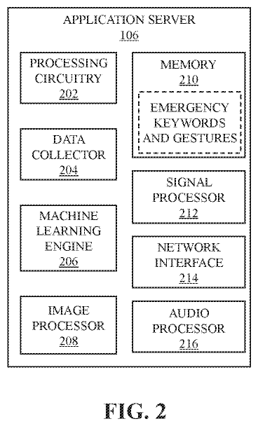

In another embodiment, the application server 106 may be configured to store medical profiles of the occupants 110 and 112 in the memory (shown in FIG. 2) thereof. The medical profile may include a medical history, list of allergies, normal vitals, or the like of the occupants 110 and 112. In one instance, the application server 106 may receive the emergency signal based on sensor data generated by the image sensor and the fitness accessory. The emergency signal may include image data and current health state data of the occupants 110 and 112. The image data may indicate state of consciousness of the occupants 110 and 112 along with other in-vehicle activities. The current health state data may indicate health conditions of the occupant 110 and the occupant 112. The application server 106 may process the image data and the current health state data to determine the severity level and the context of the in-vehicle emergency. In one example, the application server 106 may determine, based on semi-conscious state of the occupant 110 or 112 in the image data, that the in-vehicle emergency has a high-severity. Further, based on the current health state data indicating a low sugar level and semi-conscious state of the occupant 110 or 112, the application server 106 may determine the context indicative of a medical emergency. Further, the context may also indicate that due to the semi-conscious state of the occupant 110 or 112, a response team is required to reach a current location of the vehicle 102. In an alternative embodiment, the fitness accessory may transmit the current health state data to a third-party server (not shown) associated with the fitness accessory. The third-party server then processes the current health state data to detect the health conditions of the occupant 110 or 112. The third-party server may then transmit the detected health conditions to the application server 106. The application server 106 may process the detected health conditions to detect the severity level and the context of the in-vehicle emergency.

In an embodiment, the first physical factors that indicate the context of the in-vehicle emergency may include moving or at rest state of the vehicle 102, a functional state of one or more components of the vehicle 102, the current location of the vehicle 102, one or more damages caused to the vehicle 102, a speed of the vehicle 102, an acceleration or deacceleration of the vehicle 102, and a direction of movement of the vehicle 102.

In another embodiment, the second physical factors that indicate the context of the in-vehicle emergency may include a physical orientation of the occupants 110 and 112, a state of calmness or panic of the occupants 110 and 112, any physical harm caused to any of the occupants 110 and 112, and an ability or disability of any of the occupants 110 or 112 to control the vehicle 102 or follow emergency assistance guidelines and navigation assistance.

In another embodiment, the medical factors that indicate the context of the in-vehicle emergency may include a state of consciousness or unconsciousness of each occupant 110 and 112, a blood pressure of each occupant 110 and 112, a sugar level of each occupant 110 and 112, a pulse rate of each occupant 110 and 112, vitals associated with each occupant 110 and 112, and any deviation of a current state of health of each occupant 110 and 112 from the stored medical profile of the corresponding occupant 110 and 112.

In another embodiment, the infrastructure availability factors, around the current location of the vehicle 102, that indicate the context of the in-vehicle emergency may include a presence or an absence of a required response team in vicinity of the vehicle 102, an availability of resources (such as water in case of a fire in the vehicle 102, a maintenance center in case of failure of brakes of the vehicle 102, and the like) required to handle the in-vehicle emergency, or the like. The infrastructure availability factors, around the current location of the vehicle 102, that indicate the context of the in-vehicle emergency may further include a presence or an absence of passer-by individuals in the vicinity of the vehicle 102.

In another embodiment, the environmental factors associated with the geographical region of the vehicle 102 that indicate the context of the in-vehicle emergency may include a time of day, an availability or an unavailability of light, rain, thunderstorm, temperature, or the like. Such environmental factors significantly affect a time and a type of assistance that is required for resolving the in-vehicle emergency.

The application server 106 may determine the first physical factors, the second physical factors, the medical factors, the infrastructure availability factors, and the environmental factors based on analysis and processing of the sensor data. In one example, the application server 106 may determine the environmental factors based on the location data received from the GPS sensor. In another example, the application server 106 may determine the second physical parameters based on the image data received from the image sensor. In another example, the application server 106 may determine the medical parameters based on the current health state data received from the fitness accessory (shown in FIG. 3) of each occupant 110 and 112. Beneficially, taking into account the context of the in-vehicle emergency while providing the emergency assistance significantly improves efficiency and quality of the emergency assistance. Further, taking into account the context significantly reduces a probability of loss of life and resources.

In another embodiment, the application server 106 may be further configured to display the sensor data, the severity level, and the context of the in-vehicle emergency on a computing device of an emergency operator. The in-vehicle emergency may be confirmed and verified, based on the severity level and the context, by the emergency operator. In one example, the emergency operator may be a person accountable for verifying the in-vehicle emergency. Further, the emergency operator may also suggest one or more response actions for handling the in-vehicle emergency. In one embodiment, the application server 106 may be further configured to update the severity level and the context of the in-vehicle emergency based on a feedback received from the emergency operator.

Based on the severity level and the context of the in-vehicle emergency, the application server 106 may be configured to select one or more suitable responses from a plurality of responses. The plurality of responses may include actions that may be taken for handling different in-vehicle emergencies. The plurality of responses may include display of contextual text and/or graphical symbols (i.e., textual and graphical content) indicating the detected in-vehicle emergency on an external vehicular display (such as an LCD/LED screen fitted outside the vehicle 102), and communication of a contextual message, prerecorded message, an emergency signal, sensor data, or the like to the emergency contact of each occupant 110 and 112. The plurality of responses may further include display of an emergency assistance message to the occupants 110 and 112 via the user device 102a, display of the navigation assistance to the occupants 110 and 112, lock or unlock action of a central lock of the vehicle 102, speed manipulation of a driving speed of the vehicle 102, and initiation of a troubleshoot procedure of the vehicle 102.

The application server 106 may be further configured to execute one or more response actions associated with the selected responses. The application server 106 may be configured to execute the response actions by communicating an instruction to one or more components (such as, a central controller, the telematics device, the OBD device of the vehicle 102), the response team associated with the in-vehicle emergency, the occupants 110 and 112, the emergency contacts of the occupants 110 and 112, or the like.

For executing the response actions, the application server 106 may be further configured to displaying via the external vehicular display of the vehicle 102 textual and graphical content indicating the in-vehicle emergency. The textual and graphical content may be provided by any of the occupants 110 and 112 via the user device 102a. Alternatively, the application server 106 may be configured to determine the textual and graphical content based on the severity level and the context of the in-vehicle emergency. For example, in an instance of failure of brakes of the vehicle 102, the application server 106 may cause the external vehicular display to render text such as, “BRAKES FAIL, STAY AWAY” or “CALL 911”. The response actions may further include displaying the emergency assistance message on the user device 102a of the occupants 110 and 112, providing the navigation assistance to the occupants 110 and 112 on the user device 102a or a vehicle device (such as a vehicle head unit) of the vehicle 102, controlling remotely the central lock of the vehicle 102 for locking or unlocking the vehicle 102, controlling remotely the driving speed of the vehicle 102 to halt the vehicle 102, and initiating the troubleshooting of the vehicle 102. The application server 106 may be configured to initiate the troubleshooting of the vehicle 102 by activating or deactivating one or more components (such as the OBD device) of the vehicle 102. In an embodiment, the occupant 110 may not be able to drive. Therefore, the response actions may include, confirming with the occupant 112 regarding his/her ability to drive the vehicle 102. The application server 106 may be configured to perform the step of confirming with the occupant 112 by rendering an interactive interface, on the user device 102a, to enable the occupant 112 to provide the confirmation.

In an embodiment, the application server 106 may be configured to initiate an interactive voice response (IVR) call to the emergency contacts of the occupants 110 and 112. The IVR call may be initiated to provide personal and professional details about the occupants 110 and 112. Such personal and professional details may be useful for providing an efficient emergency assistance to the occupants 110 and 112. In another embodiment, the application server 106 may be configured to communicate a contextual message or a pre-recorded message to the emergency contacts of the occupants 110 and 112. The pre-recorded message may be voice data pre-recorded by the corresponding occupant 110 and 112 and may be associated with the context of the in-vehicle emergency.

In one embodiment, after the detection of the in-vehicle emergency, the application server 106 may determine that a response time of the emergency assistance for handling the in-vehicle emergency may be greater than a threshold response time (for example, 5 minutes, 10 minutes, 15 minutes, and so on). In such scenarios, the application server 106 may generate a set of alert signals for informing passer-by individuals and vehicles regarding the in-vehicle emergency of the vehicle 102. For example, the application server 106 may cause a speaker or siren (not shown) of the vehicle 102 that may be externally installed such as on a roof-top of the vehicle 102 to play an audio message. In another example, the application server 106 may generate an emergency notification based on the severity level and the context of the detected in-vehicle emergency. The emergency notification may be represented by means of a visual signal, a light signal, a textual or graphical signal, or any combination thereof via the external vehicular display of the vehicle 102.

In one embodiment, the application server 106 may be a remote server that is external to the vehicle 102. In such a scenario, the application server 106 may communicate with the vehicle 102 and the user device 102a via the communication network 108 for handling the in-vehicle emergencies. In another embodiment, the application server 106 may be implemented as a local server inside the vehicle 102 for in-vehicle emergency assistance. The application server 106, when implemented as the local server, may communicate with different components of the vehicle 102 and the user device 102a via a controlled area network (CAN) of the vehicle 102.

In an embodiment, when the user device 102a may be offline and may not be able to communicate with the application server 106 via the communication network 108. In such embodiment, the mobile application may operate in an offline mode. While working in the offline mode, the mobile application may be configured to use a mobile network of the user device 102a to contact the emergency contacts and the response team. The mobile application may also be configured to initiate an audio system of the vehicle 102 to announce the in-vehicle emergency to passer-by individuals and other vehicles. Further, the mobile application may be configured to cause the external vehicular display of the vehicle 102 to display an emergency text (such as help, call 911, and the like). The term “audio system” will be described in detail in conjunction with description of FIG. 3. It will be apparent to a person of ordinary skill in the art that handling the emergency response is same as handling the in-vehicle emergency.

FIG. 2 is a block diagram that illustrates the application server 106, in accordance with an exemplary embodiment of the disclosure. The application server 106 may include processing circuitry 202, a data collector 204, a machine learning engine 206, an image processor 208, the memory (hereinafter, referred to and designated as “the memory 210”), a signal processor 212, a network interface 214, and an audio processor 216.

The processing circuitry 202 may include suitable logic, circuitry, interfaces, and/or code, executable by the circuitry, that may be configured to execute the instructions stored in the memory 210 to perform various operations for handling the in-vehicle emergency. The processing circuitry 202 may be configured to perform various operations associated with data collection and data processing. The processing circuitry 202 may be implemented by one or more processors, such as, but not limited to, an application-specific integrated circuit (ASIC) processor, a reduced instruction set computing (RISC) processor, a complex instruction set computing (CISC) processor, and a field-programmable gate array (FPGA) processor. The one or more processors may also correspond to central processing units (CPUs), graphics processing units (GPUs), network processing units (NPUs), digital signal processors (DSPs), or the like. It will be apparent to a person of ordinary skill in the art that the processing circuitry 202 may be compatible with multiple operating systems. The processing circuitry 202 may be configured to receive the emergency signal from the vehicle 102 or the user device 102a. The processing circuitry 202 may be further configured to activate the plurality of sensors and the fitness accessory in the vehicle 102 for sensor data collection. The processing circuitry 202 may be further configured to process the collected sensor data and the collected current health state data to detect the in-vehicle emergency. The processing circuitry 202 may be further configured to determine a severity level and a context associated with the detected in-vehicle emergency based on the collected sensor data. The processing circuitry 202 may be further configured to select, from the plurality of responses, one or more responses to handle the detected in-vehicle emergency. The one or more responses are selected based on the determined severity level and the determined context. The processing circuitry 202 may be further configured to execute one or more response actions (as described in the foregoing description of FIG. 1) based on the selected one or more responses.

The data collector 204 may include suitable logic, circuitry, interfaces, and/or code, executable by the circuitry, that may be configured to execute the instructions stored in the memory 210 to collect data such as the sensor data from the plurality of sensors, the contextual text, the current health state data from the fitness accessory, or the like. In one embodiment, the data collector 204 may be activated by the processing circuitry 202 based on the reception of the emergency signal. In another embodiment, the data collector 204 may be configured to collect data from the vehicle 102 and the user device 102a periodically (such as, after 2 minutes, 4 minutes, 6 minutes, or the like).

The machine learning engine 206 may include suitable logic, circuitry, interfaces, and/or code, executable by the circuitry, that may be configured to perform one or more operations for analyzing the sensor data and other information associated with the historical in-vehicle emergencies. Further, the machine learning engine 206 may deduce one or more rules for handling the in-vehicle emergency based on the analysis of the sensor data, the severity level, and the context of the historical in-vehicle emergencies. In other words, the machine learning engine 206 may be configured to capture a trend or a pattern associated with the historical in-vehicle emergencies and deduce rules for handling future in-vehicle emergencies more efficiently. In an example, the machine learning engine 206 may have observed in the past that the occupant 112 has a habit of saying “OH GOD, HELP ME”. Therefore, the machine learning engine 206 may have deduced a rule to eliminate such false positive in-vehicle emergencies initiated due to utterance of the emergency keyword “HELP” by the occupant 112.

The image processor 208 may include suitable logic, circuitry, interfaces, and/or code, executable by the circuitry, that may be configured to perform one or more operations for analyzing the image data received from the image sensor. The image processor 208 may be configured to apply one or more image processing algorithms or techniques that are known in the art to analyze the image data. The image processor 208 may be further configured to optimize the image data and filter noise therein to obtain clear and filtered images. Based on the filtered images, the image processor 208 may identify one or more in-vehicle actions, state of consciousness of the occupants 110 and 112, presence or absence of fire, smoke, or water inside the vehicle 102, a physical altercation with a third party or between the occupants 110 and 112, or the like.

The memory 210 may include suitable logic, circuitry, and interfaces that may be configured to store one or more instructions which when executed by the processing circuitry 202 cause the processing circuitry 202 to perform various operations for handling emergency responses for resolving in-vehicle emergencies. The memory 210 may be accessible by the processing circuitry 202, the data collector 204, the machine learning engine 206, the image processor 208, the signal processor 212, and the audio processor 216. Examples of the memory 210 may include, but are not limited to, a random-access memory (RAM), a read only memory (ROM), a removable storage drive, a hard disk drive (HDD), a flash memory, a solid-state memory, or the like. It will be apparent to a person skilled in the art that the scope of the disclosure is not limited to realizing the memory 210 in the application server 106, as described herein. In another embodiment, the memory 210 may be realized in form of the database server 104 or a cloud storage working in conjunction with the application server 106, without departing from the scope of the disclosure.

In an embodiment, the memory 210 may store the plurality of emergency keywords that indicate the plurality of in-vehicle emergencies. In one embodiment, the machine learning engine 206 may be configured to define and identify the plurality emergency keywords. The machine learning engine 206 may further update the plurality of emergency keywords based on the sensor data (i.e., words recorded by the microphone), the severity level, and the context of historical in-vehicle emergencies.

The plurality of emergency keywords may be downloaded in a memory of the user device 102a upon installation or first time use of the mobile application on the user device 102a. In one embodiment, the user device 102a may enable the occupants 110 or 112 to define personalized emergency keywords. Examples of the plurality of emergency keywords may include, but are not limited to, “help”, “fire”, “call 911”, or the like. In one instance, the occupant 110 or the occupant 112 may utter one or more keywords. The microphone in the vehicle 102a or the user device 102a may capture the one or more keywords and when the uttered keywords are present in the plurality of emergency keywords, the mobile application generates the emergency signal. In one embodiment, the activation and deactivation of the microphone for capturing and listening the in-vehicle speech data is controlled based on a consent of the occupants 110 and 112.

In another embodiment, the memory 210 may store the plurality of gestures indicating the plurality of in-vehicle emergencies. In one embodiment, the machine learning engine 206 may be configured to define and identify the plurality of gestures. The machine learning engine 206 may further update the plurality of gestures based on the sensors data (i.e., image captured by the image sensor), the severity level, and the context of historical in-vehicle emergencies.

The plurality of gestures may be downloaded in the memory of the user device 102a upon installation or first time use of the mobile application on the user device 102a. In one embodiment, the user device 102a may enable the occupants 110 or 112 to define personalized gestures to indicate different emergencies. In such embodiment, the emergency signal may be received based on detection of the action of the occupants 110 or 112 that results in one or more gestures of the plurality of gestures. In one example, the plurality of gestures may include at least one hand of the occupant 110 or 112 covering his eyes and head. In one instance, the occupant 110 or the occupant 112 performs an action of crossing hands in front of face in such a way that his hands cover eyes and head. Therefore, the image sensor detects a gesture from the plurality of gestures indicating an occurrence of one of the plurality of in-vehicle emergencies. Upon detection of such gesture, based on the image data captured by the image sensor, the mobile application generates the emergency signal. In one embodiment, the activation and deactivation of the image sensor for capturing in-vehicle activities is controlled based on a consent of the occupants 110 and 112.

The signal processor 212 may include suitable logic, circuitry, interfaces, and/or code, executable by the circuitry, that may be configured to perform one or more operations for processing the received emergency signal and the sensor data. The signal processor 212 may apply one or more signal processing techniques known in the art to process the emergency signal and the sensor data. The signal processor 212 may analyze the emergency signal and the sensor data to determine strength of the emergency signal, content of the emergency signal, a source or trigger of the emergency signal, or the like.

The network interface 214 may include suitable logic, circuitry, interfaces, and/or code, executable by the circuitry, that may be configured to enable the application server 106 to communicate with the vehicle 102, the user device 102a, the emergency contacts of the occupants 110 and 112, the response teams, or the like. The network interface 214 may be configured to receive the emergency signal from the vehicle 102 or the user device 102a. The network interface 214 may be further configured to communicate one or more notifications or instructions to the response teams, the emergency contacts, the user device 102a, and the vehicle 102. The network interface 214 may be implemented as a hardware, software, firmware, or a combination thereof. Examples of the network interface 214 may include a network interface card, a physical port, a network interface device, an antenna, a radio frequency transceiver, a wireless transceiver, an Ethernet port, a universal serial bus (USB) port, or the like.

The audio processor 216 may include suitable logic, circuitry, interfaces, and/or code, executable by the circuitry, that may be configured to perform one or more operations for analyzing the speech data recorded or captured by the microphone of the vehicle 102. The speech data is recorded or captured by the microphone based on the consent of the occupants 110 and 112. The audio processor 216 may be configured to apply one or more natural language processing (NLP) algorithms known in the art to analyze the speech data. The audio processor 216 may be further configured to optimize the speech data and filter noise therein to obtain a clear and filtered audio. Based on the filtered audio, the audio processor 216 may identify one or more emergency keywords or phrases uttered by the occupants 110 and 112, the context of the speech data, distress level of the speech data, pitch and count of the emergency keywords or phrases uttered by the occupants 110 and 112, and the state of consciousness of the occupants 110 and 112. Based on the filtered audio, the audio processor 216 may further identify the state of calmness or panic of the occupants 110 and 112, a presence or an absence of another occupant in the vehicle 102, and an altercation between the occupants 110 and 112 or between any of the occupant 110 or 112 and another individual.

It will be apparent to a person skilled in the art that the application server 106 shown in FIG. 2 is an exemplary illustration, and in different embodiments the application server 106 may include additional or different components configured to perform similar or different operations without departing from the scope of the disclosure.

FIG. 3 is a schematic diagram that illustrates an exemplary scenario of a top view 300 of the vehicle 102, in accordance with an exemplary embodiment of the disclosure. The vehicle 102 may have the occupant 110 sitting on a driver seat 302 and the occupant 112 sitting on a backseat 304. The vehicle 102 is further associated with the user device 102a, a passenger device 306, the emergency input interface such as physical emergency buttons 308a and 308b, the microphone 310, the image sensor 312, the impact sensor 314, the temperature sensor 316, the smoke sensor 318, the pressure sensor 320, the speed sensor 322, the proximity sensor 324, a first fitness accessory 325a of the occupant 110, and a second fitness accessory 325b of the occupant 112. The vehicle 102 may further include the external vehicular display 326 installed on a back side of the vehicle 102. The vehicle 102 may further include the global positioning system (GPS) sensor 328. In an embodiment, the user device 102a, the passenger device 306, the physical emergency buttons 308a and 308b, the microphone 310, the image sensor 312, the impact sensor 314, the temperature sensor 316, the smoke sensor 318, the pressure sensor 320, the speed sensor 322, the proximity sensor 324, the first fitness accessory 325a, the second fitness accessory 325b, the external vehicular display 326, and the GPS sensor 328 may be communicatively coupled to each other by means of the vehicle device (not shown) of the vehicle 102. In another embodiment, the user device 102a, the passenger device 306, the physical emergency buttons 308a and 308b, the microphone 310, the image sensor 312, the impact sensor 314, the temperature sensor 316, the smoke sensor 318, the pressure sensor 320, the speed sensor 322, the proximity sensor 324, the first fitness accessory 325a, the second fitness accessory 325b, the external vehicular display 326, and the GPS sensors 328 may be communicatively coupled to the application server 106 via the communication network 108. Also, the vehicle device of the vehicle 102 may be communicatively coupled to the application server 106 via the communication network 108. For the sake of ongoing description, it is assumed that the user device 102a is a dedicated communication device of the occupant 110 and the passenger device 306 is a dedicated communication device of the occupant 112.

It may be apparent to a person skilled in the art that one or more operations performed by the user device 102a (as described in the foregoing description of FIG. 1) may also be performed by the passenger device 306 in different embodiments of the disclosure.

Each of the user device 102a and the passenger device 306 includes suitable logic, circuitry, and/or interfaces that are operable to perform one or more operations for initiating emergency assistance for an in-vehicle emergency. The user device 102a and the passenger device 306 may have the mobile application installed therein. The mobile application may facilitate mobile application-based emergency buttons, in the user device 102a and the passenger device 306, for use by the occupant 110 and the occupant 112, respectively. When the occupant 110 presses the application-based emergency button on the user device 102a, the emergency signal may be communicated to the application server 106 indicating a possible occurrence of an in-vehicle emergency. Similarly, when the occupant 112 presses the application-based emergency button on the passenger device 306, the emergency signal may be transmitted to the application server 106 indicating the possible occurrence of an in-vehicle emergency. In one embodiment, the mobile application is a ride-booking mobile application. In such a scenario, if the vehicle 102 is booked by the occupant 112 using the mobile application, pressing of the application-based emergency button on the passenger device 306 after the initiation of the ride indicates the possible occurrence of an in-vehicle emergency.

The physical emergency buttons 308a and 308b are switches (such as electronic switches, mechanical switches, or any combination thereof) installed in the vicinity of the occupant 110 and the occupant 112, respectively. The physical emergency buttons 308a and 308b may facilitate easy access to the respective occupants 110 and 112 to initiate emergency assistance in an event of a safety concern associated with the vehicle 102, the occupant 110, and/or the occupant 112. When the occupant 110 presses the physical emergency button 308a, the emergency signal may be communicated to the application server 106 indicating the possible occurrence of the in-vehicle emergency. Similarly, when the occupant 112 presses the physical emergency button 308b, the emergency signal may be transmitted to the application server 106 indicating the possible occurrence of the in-vehicle emergency.

The microphone 310 includes suitable logic, circuitry, and/or interfaces that are operable to execute one or more instructions for performing one or more operations to initiate the emergency assistance. In an embodiment, the microphone 310 may be an audio-capturing and recording device that is installed in the vehicle 102 for capturing and recording in-vehicle audio information (i.e., the speech data) including various emergency keywords uttered by the occupants 110 and/or 112. The microphone 310 may be configured to record the speech data including the emergency keywords uttered by the occupants 110 and 112. The microphone 310 may be further configured to transmit (or communicate) the speech data to the user device 102a or the passenger device 306 for further analysis and processing. The user device 102a and the passenger device 306 may be configured to process the speech data to identify the uttered emergency keywords. In another embodiment, the microphone 310 may transmit the recorded speech data to the OBD device of the vehicle 102. The OBD device may be configured to identify the uttered keywords. Based on identification of the uttered keywords, the emergency signal is generated and communicated to the application server 106. Further, the user device 102a, the passenger device 306, or the OBD device may identify the uttered emergency keywords from the speech data based on a comparison of the speech data with the downloaded plurality of emergency keywords.

The image sensor 312 includes suitable logic, circuitry, and/or interfaces that are operable to execute one or more operations to initiate the emergency assistance. In an embodiment, the image sensor 312 is a camera device that is installed in the vehicle 102 for capturing and recording in-vehicle image and video information (i.e., the image data and video data) of the vehicle 102 that may or may not include the occupant 110 and/or the occupant 112. In an exemplary embodiment, the image sensor 312 may be installed on a windshield 324 of the vehicle 102. Further, the image sensor 312 may be oriented to face inside the vehicle 102 for capturing and recording the in-vehicle image and video information corresponding to various in-vehicle activities. In another embodiment, the image sensor 312 may be installed on other vehicle parts of the vehicle 102, such as a side mirror 326, a first door (not shown), or the like, for capturing and recording the in-vehicle image and video information. In an embodiment, the image sensor 326 may have a 360-degree view for capturing the image data. In such embodiment, the image sensor 326 may capture a view outside the vehicle 102 as well the in-vehicle activities. The in-vehicle activities may include, but are not limited to, one or more activities related to the occupants 110 and 112, or other objects inside the vehicle 102. In an embodiment, the image sensor 312 may be activated to capture and record the in-vehicle image and video information based on an activation signal communicated by the application server 106. The application server 106 may generate and communicate an activation signal for activating the plurality of sensors upon reception of the emergency signal. In another embodiment, the image sensor 312 may be automatically activated to capture and record the in-vehicle image and video information in the event of the in-vehicle emergency. In another embodiment, the occupants 110 and 112 may activate the image sensor 312 via the mobile application or by manually pressing an ON button present on a body of the image sensor 312. In another embodiment, the image sensor 312 may be automatically activated upon the consent of occupants 110 and 112 at the start of the ride so as to capture in-vehicle activities indicating one or more gestures from the downloaded plurality of gestures.

The impact sensor 314, the temperature sensor 316, and the smoke sensor 320 are installed in the vehicle 102 to detect and monitor the health conditions of the vehicle 102 in real-time. In an embodiment, the impact sensor 314 may be installed on outer body of the vehicle 102. The impact sensor 314 may be configured to detect sudden shock, impact, or vibration caused to a surface of the vehicle 102. Such shock, impact, or vibration may be a result of collision and may be indicative of the in-vehicle emergency. The temperature sensor 316 may be installed inside the vehicle 102 to detect a minimum and a maximum temperature inside the vehicle 102. Detection of a high temperature inside the vehicle 102 may indicate failure of air-conditioner (AC) of the vehicle 102 or an instance of fire caused in the vehicle 102. Detection of a low temperature may indicate failure of a heater of the vehicle 102. A low temperature inside the vehicle 102 for very long hours may indicate that the vehicle 102 may be stranded and may not be in-use over a period of time. The smoke sensor 320 may be installed inside and/or on the outer surface of the vehicle 102. The smoke sensor 320 may detect a presence of smoke caused due to short circuit or fire caused in the vehicle 102.

The GPS sensor 328 may be configured to detect the current location of the vehicle 102. The GPS sensor 328 may or may not require activation by the application server 106. In an embodiment the GPS sensor 328 may continuously track the current location of the vehicle 102. The GPS sensor 328 may be further configured to communicate the current location of the vehicle 102 when prompted or instructed by the application server 106. In one example, the GPS sensor 328 may communicate the current location to the mobile application hosted on the user device 102a and the passenger device 306. The user device 102a and the passenger device 306 may communicate the current location to the application server 106.

In an embodiment, the plurality of sensors may further include, but are not limited to, an airbag sensor, a gyroscope sensor, an accelerometer sensor, and an OBD sensor. The airbag sensor is a sensor that detects an impact or collision of the vehicle 102 with an on-road object (such as a pedestrian, an animal, a road-divider, a non-drivable area, a rock, another vehicle, or the like) and generates airbag data. In an embodiment, the application server 106 is configured to deploy an airbag of the vehicle 102 based on the airbag data. The gyroscope sensor is a sensor that detects and measures an angular velocity of the vehicle 102 and generates gyroscope data indicating an angular velocity of the vehicle 102. The accelerometer sensor is a sensor that detects and measures acceleration of the vehicle 102 and generates accelerometer data indicating the acceleration of the vehicle 102. The accelerometer data may be further indicative of swinging, tilting, rotating, and shaking of the vehicle 102. The OBD sensor is a sensor (or a group of sensors) that detects and measures various forms of diagnostic data associated with the vehicle 102 and generates OBD data. The OBD data may include data, such as tire pressure, braking frequency, fuel level, temperature, speed, airflow rate, coolant temperature, spark advance, oxygen sensor test results, and the like, associated with the vehicle 102. In an embodiment, the sensor data may further include one of the airbag data, the gyroscope data, the accelerometer data, and the OBD data.

In an embodiment, the plurality of sensors may further include various sensors that are installed in the vehicle 102 (for example, in one or more seats, doors, or windshields of the vehicle 102) to detect and monitor the health conditions of the occupants 110 and 112, in real-time. Such sensors may include, but are not limited to, the heart rate sensor, the blood pressure sensor, the body sensor, and the IR sensor. The heart rate sensor and the blood pressure sensor may measure and monitor the heart rate and the blood pressure of the occupants 110 and 112. The body sensor measures and monitors various vital signs of the occupants 110 and 112. The IR sensor measures and monitors body temperatures of the occupants 110 and 112. The various sensors the detect and monitor the health conditions of the occupants 110 and 112 may generate the current health state data of the occupants 110 and 112.

The fitness accessories 325a and 325b includes suitable logic, circuitry, and/or interfaces that are operable to perform one or more operations. In an embodiment, the fitness accessories 325a and 325b are wearable electronic devices associated with the occupant 110 and the occupant 112, respectively. A fitness accessory may correspond to the fitness watch, the fitness band, or the like. The fitness accessories 325a and 325b may detect and monitor the real-time health conditions of the occupant 110 and the occupant 112, respectively.

The vehicle 102 may further include the audio system (not shown) that includes suitable logic, circuitry, and/or interfaces that are operable to execute one or more instructions stored in its memory to perform one or more operations. The audio system may include an audio device such as an audio speaker, a horn, a siren, or the like. In one embodiment, the audio system receives an instruction associated with execution of the response actions from the application server 106 (alternatively, from the vehicle device of the vehicle 102 or the mobile application) and turns ON the horn or the siren to alert the occupants 110 and 112, the passer-by individuals, the near-by vehicles, or the like. In another embodiment, the audio system receives the instruction from the application server 106 (alternatively, from the vehicle device of the vehicle 102 or the mobile application) and turns ON the audio speaker to output an audio recording indicating the in-vehicle emergency that may alert the occupants 110 and 112, the passer-by individuals, the near-by vehicles, or the like.

The external vehicular display 326 includes suitable logic, circuitry, and/or interfaces that are operable to execute one or more instructions stored in its memory to perform one or more operations. In an embodiment, the external vehicular display 326 receives the instruction from the application server 106 (alternatively, from the vehicle device of the vehicle 102 or the mobile application) and displays the emergency message to alert the passer-by individuals, the near-by vehicles, or the like. The external vehicular display 326 may use backlit light emitting diodes (LEDs), fonts, animations, or the like, for ensuring legible reading to the passer-by individuals, the near-by vehicles, or the like. Although FIG. 3 illustrates the external vehicular display 326 installed on the windshield 330 of the vehicle 102, it will be apparent to a person skilled in the art that the external vehicular display 326 may be installed on a rooftop of the vehicle 102, sides of the vehicle 102, or the like. Further, the external vehicular display 326 may include one or more displays, such as, a front display, a rear display, a first side display, a second side display, or any combination thereof, for displaying the emergency message and/or the in-vehicle image and video information.

It will be apparent to a person of ordinary skill in the art that the sensors that monitor the health of the vehicle 102 and the occupants 110 and 112 may be activated automatically on the use of the vehicle 102.

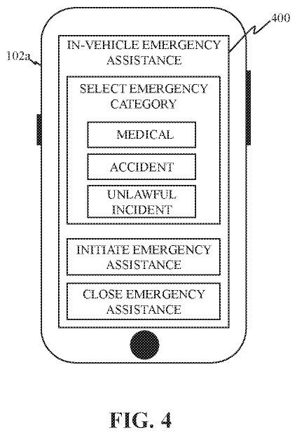

FIG. 4 is a schematic diagram that illustrates a user interface 400 rendered by the application server 106 on the user device 102a, in accordance with an exemplary embodiment of the disclosure. The user interface 400 facilitates reception of an emergency input via touch-based input buttons. Based on the emergency input, mobile application-based emergency signal is generated and communicated to the application server 106. The user interface 400 provides various options for indicating occurrence of the in-vehicle emergency. The user interface 400 also facilitates selection of a category of the in-vehicle emergency. The category of the in-vehicle emergency may refer to a type of emergency. The categories of the in-vehicle emergency may be a medical emergency, an accidental emergency, and an unlawful activity emergency. The category selection may be provided based on selection of one of “MEDICAL”, “ACCIDENT”, and “UNLAWFUL INCIDENT” options via the user interface 400.

The user interface 400 further facilitates an option for initiating the in-vehicle emergency assistance. The user interface 400 enables the occupant 110 to select the “INITIATE EMERGENCY ASSISTANCE” option. Based on selection of the “INITIATE EMERGENCY ASSISTANCE” option, the emergency signal is generated and communicated to the application server 106. Further, the user interface 400 may also provide an option “CLOSE EMERGENCY ASSISTANCE” for terminating the in-vehicle emergency assistance in case of one of a false positive, when emergency assistance is initiated by mistake, or when the emergency assistance is no longer required.

In an embodiment, the user interface 400 may also display a confirmation message for confirming occurrence of the in-vehicle emergency when the emergency signal is generated based on the sensor data. Beneficially, such confirmation reduces a probability of false positive incidents and hence saves time, cost, and effort required for the emergency assistance. The false positive may be a case where an in-vehicle emergency, that is detected by the application server 106, does not exist in reality.

In an embodiment, the user interface 400 may also display a notification when an initiation of the emergency assistance may have got cancelled based on the collected sensor data. The occupant 110 may choose to go ahead with the emergency assistance via the mobile-based application in case of a false negative. The false negative may be a case of where an actual in-vehicle emergency is discarded by the application server 106 based on an invalidation of the sensor data. In an embodiment, the user interface 400 may be configured to display an actual central lock status of the vehicle 102 via the user device 102a.

It will be apparent to a person of ordinary skill in the art that a user interface similar to the user interface 400 may be rendered on the passenger device 306 for enabling the occupant 112 to initiate emergency assistance, without departing from the scope of the disclosure.

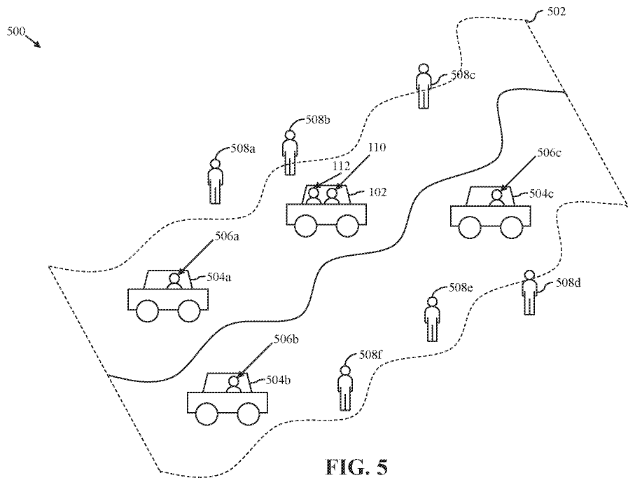

FIG. 5 is a block diagram that illustrates an exemplary environment 500 for in-vehicle emergency detection and response handling, in accordance with an exemplary embodiment of the disclosure. The environment 500 illustrates a road 502 along which the occupant 110 is driving the vehicle 102. The environment 500 further illustrates vehicles 504a-504c that are being driven by drivers 506a-506c, respectively, along the road 502. The vehicles 504a-504c are in the vicinity of the vehicle 102 (i.e., within a defined radius of a current location of the vehicle 102). The environment 500 further shows passer-by individuals 508a-508f within a first distance of the vehicle 102 (i.e., within a defined radius of a current location of the vehicle 102). The first distance may be a threshold distance that is in vicinity of the vehicle 102.

In an embodiment, the occupant 110 is driving the vehicle 102 and the occupant 112 is occupying the backseat 304 of the vehicle 102. When the vehicle 102 is in motion, there may be an occurrence of an in-vehicle emergency inside the vehicle 102. For the sake of ongoing description, it is assumed that the in-vehicle emergency corresponds to a scenario in which the occupant 112 may be assaulting or attacking the occupant 110.

When the occupant 110 is under attack, the occupant 110 may press the physical emergency button 308a. An emergency signal is generated and transmitted to the application server 106. Upon reception of the emergency signal, the application server 106 activates the plurality of sensors and collects the sensor data. Further, the application server 106 may also communicate a feedback signal to the occupant 110 by glowing one or more lights embedded with the physical emergency button 308a to indicate a reception of the emergency signal. The application server 106 may process the sensor data (i.e., the image data and the speech data) to detect the in-vehicle emergency. The application server 106 may further process the image data and identify a knife in a hand of the passenger 112. Therefore, the application server 106 may determine a high severity level of the in-vehicle emergency as life of the occupant 110 is at stake. The application server 106 may further analyze the speech data to determine a distress level of the occupant 110. The distress level may be high when one of shouting, crying, verbal abuse, or the like is detected in the speech data. The distress level may be further determined as a function of the pitch and the count of emergency keywords detected in the speech data. In the present exemplary scenario, based on the analysis of the speech data, the application server 106 may detect that the occupant 110 is shouting “DON'T KILL ME”. Therefore, the application server 106 may determine that the context of the in-vehicle emergency may be physical assault which is further related to the unlawful incident as well as the medical emergency. The context may further indicate that the occupant 110 is scared and may not be able to handle the situation well. Based on the detected in-vehicle emergency and the determined severity level and the context of the in-vehicle emergency, the application server 106 selects one or more responses from the plurality of responses. The selected responses may be associated with the response actions such as informing nearest police station, halting the vehicle 102, unlocking the central lock of the vehicle 102, and displaying textual and graphical content (for example, an emergency message such as “HELP”) on the external vehicular display 326. In one example, if the context of the detected in-vehicle emergency indicates that the occupant 110 is injured, calling for an ambulance may be one of the response actions executed by the application server 106.

The application server 106 may further communicate with the response teams (i.e., the police team of the nearest police station and a medical team), one or more components of the vehicle 102 or the user device 102a to execute the selected response actions. In one embodiment, although the central lock is unlocked by the application server 106, the unlock status of the central lock may be only visible to the occupant 110 on the user device 102a. Thus, causing a deception of a false central door lock for the occupant 112.

Further, the application server 106 may retrieve the emergency contact associated with the occupant 110 and communicate the emergency message and real-time data (i.e., the live feed of the in-vehicle activities and the current location of the vehicle 102) to the emergency contact and the response teams. The application server 106 may further transmit the captured real-time data to the response teams that are in vicinity of the current location of the vehicle 102. The response teams may track the vehicle 102 based on the current location and visualize the real-time conditions inside the vehicle 102 based on the live feed. Further, to provide additional assistance to the occupant 110, the application server 106 may be configured to reduce the speed of the vehicle 102 and/or halt the vehicle 102 in a way that it deceives the occupant 112 of vehicle breakdown. In other words, the application server 106 may remotely control one or more components of the vehicle 102 to simulate a false vehicle breakdown for the vehicle 102. Further, the application server 106 may also be configured to enable a siren installed in the vehicle 102 in order to make the drivers 506a-506c and the passer-by individuals 508a-508f aware of the in-vehicle emergency. With such effective and efficient way of communication, the various entities (such as the drivers 506a-506c, the passer-by individuals 508a-508f, or the response teams) may reach the current location to help the driver 110.

In an embodiment, the microphone 310 may be passively enabled or activated. When the microphone 310 is passively enabled, the microphone 310 may be continuously listening to one or more words uttered by the occupants 110 and 112. Further, the passively enabled microphone 310 may be configured to transmit the speech data to the mobile application. The mobile application, based on identification of one or more emergency keywords in the speech data, may communicate the emergency signal to the application server 106. Further, a consent of at least one of the occupants 110 and 112 may be required for passively enabling the microphone 310.

In one instance, the occupant 110 may not be able to press the physical emergency button 308a while being attacked by the occupant 112. In such instance, the speech data including the words “DON'T KILL ME” uttered by the occupant 110 may be communicated to the user device 102a by the passively activated microphone 310. Based on identification of an emergency keyword “KILL” in the speech data, the mobile application may initiate (or generate) the emergency signal and communicate the emergency signal to the application server 106.

In an embodiment, the image sensor 312 may be passively enabled. When the image sensor 312 is passively enabled, the image sensor 312 may continuously capture one or more in-vehicle activities. Further, the passively enabled image sensor 312 may be configured to transmit the image data to the mobile application. The mobile application, based on identification of one or more gestures from the plurality of gestures indicating the in-vehicle emergency, may communicate the emergency signal to the application server 106. Further, a consent of the occupants 110 and 112 may be required for passively enabling the image sensor 312.

In one instance, the occupant 110 may not be able to press the physical emergency button 308a or utter one or more emergency keywords while being attacked by the occupant 112. In such instance, the image data indicating the occupant 110 with folded hands and bent neck may be communicated to the user device 102a by the image sensor 312. Based on identification of the gesture “folded hands and bent neck” indicating the in-vehicle emergency, the mobile application may initiate (or generate) the emergency signal and communicate the emergency signal to the application server 106.

In another embodiment, the operations of the passively enabled image sensor 312 and microphone 310 may be implemented by a camera and a microphone of the user device 102a and/or the passenger device 306, without deviating from the scope of the disclosure.

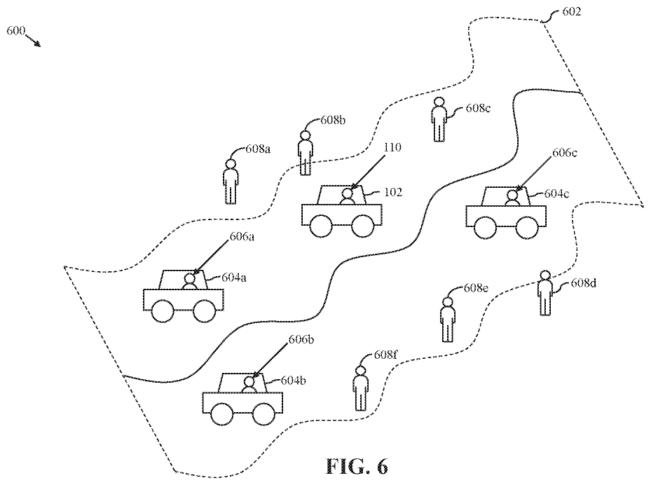

FIG. 6 is a block diagram that illustrates another exemplary environment 600 for in-vehicle emergency detection and response handling, in accordance with an exemplary embodiment of the disclosure. The environment 600 shows a road 602 along which the occupant 110 is driving the vehicle 102. The environment 600 further shows vehicles 604a-604c that are being driven by drivers 606a-606c, respectively, along the road 602. The vehicles 604a-604c are in the vicinity of the vehicle 102 (i.e., within a defined radius of the current location of the vehicle 102). The environment 600 further shows passer-by individuals 608a-608f in the vicinity of the vehicle 102 (i.e., within a defined radius of the current location of the vehicle 102).

In an embodiment, the occupant 110 is driving the vehicle 102 and is the sole occupant of the vehicle 102. When the vehicle 102 is in motion, there may be an occurrence of an in-vehicle emergency inside the vehicle 102. For the sake of ongoing description, it is assumed that the emergency corresponds to a scenario in which the occupant 110 is suffering a heart attack.

The emergency signal may be generated based on the sensor data recorded by the fitness accessory 308a of the occupant 110. Based on the received emergency signal, the application server 106 may be configured to activate the plurality of sensors. The application server 106 may further collect sensor data from the plurality of sensors. The collected sensor data may include the image data, the speech data, the current health state data, and the current location of the vehicle 102. The application server 106 may process the sensor data to detect the in-vehicle emergency. Further, based on the image data and the speech data the application server 106 may determine that the occupant 110 is unconscious and not able to drive or call for help. Further, based on the current health state data the application server 106 may determine that a pulse rate of the occupant 110 is very low. Therefore, the application server 106 may determine that the severity level of the in-vehicle emergency may be high as life of the occupant 110 is at stake and the context may include that the driver is unconscious, alone, and needs urgent medical attention.

Based on the detected in-vehicle emergency and the determined severity level and the context of the in-vehicle emergency, the application server 106 selects the one or more responses from the plurality of responses. The selected responses may include response actions such as informing nearest hospital, calling for an ambulance, halting the vehicle 102, unlocking the central lock of the vehicle 102, displaying textual and graphical content (for example, an emergency message such as “NEED HELP”) on the external vehicular display 326, playing a voice message via the audio system of the vehicle 102. The application server 106 may communicate with the response teams (i.e., doctors of the nearest police station and an ambulance operator), one or more components of the vehicle 102, and the user device 102a to execute the selected response actions.

Further, the application server 106 may retrieve the emergency contact associated with the occupant 110 and communicate the emergency message, the pre-recorded voice message, and real-time data to the emergency contacts and the response teams. The application server 106 may further transmit the emergency message and the current location data to the response teams that are in vicinity of the current location of the vehicle 102. The response teams may track the vehicle 102 based on the current location and visualize real-time conditions inside the vehicle 102 using the image sensor 312 inside the vehicle 102. Further, to provide additional assistance to the occupant 110, the application server 106 may gradually reduce the speed of the vehicle 102 and stop it within a predefined distance. With such effective and efficient way of communication, the various entities (such as the drivers 606a-606c, the passer-by individuals 608a-608f, or the response teams) may reach the incident location to help the occupant 110.

In an embodiment, the application server 106 is further configured to communicate the emergency message to one or more user device associated with the drivers 706a-706c and the passer-by individuals 708a-708f.

In an embodiment, when the occupant 110 is not the sole occupant of the vehicle 102 and the occupant 112 is also sitting in the backseat 304 of the vehicle 102. Upon determining the severity level and the context of the in-vehicle emergency, the application server 106 may be configured to communicate (as the one or more response actions) driving assistance and navigation assistance to the occupant 112 via the user device 102a or the passenger device 306.

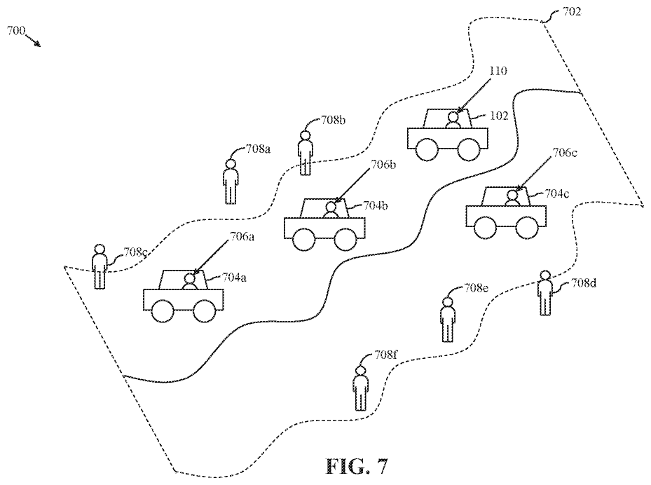

FIG. 7 is a block diagram that illustrates another exemplary environment 700 for in-vehicle emergency detection and response handling, in accordance with an exemplary embodiment of the disclosure. The environment 700 shows the road 702 along which the occupant 110 is driving the vehicle 102. The environment 700 further shows the vehicles 704a-704c that are being driven by the drivers 706a-706c, respectively, along the road 702. The vehicles 704a and 704b are positioned ahead of the vehicle 102. The environment 700 further shows passer-by individuals 708c and 708b are walking on the road 702 and are positioned ahead of the vehicle 102.

In an embodiment, when the vehicle 102 is in motion, there may be an occurrence of an in-vehicle emergency associated with the vehicle 102. For the sake of ongoing description, it is assumed that the emergency incident corresponds to a scenario in which brakes of the vehicle 102 have failed.

An emergency signal may be generated based on the sensor data recorded by the speed sensor 322 of the vehicle 102. Based on the received emergency signal, the application server 106 may be configured to activate the plurality of sensors. The application server 106 may further collect the sensor data from the plurality of sensors. The collected sensor data may include the image data, the speech data, current location data, smoke data, and temperature data of the vehicle 102. The application server 106 may detect the in-vehicle emergency by processing the sensor data. Further, based on the image data and the speech data the application server 106 may determine that the occupant 110 is alone, conscious and is able to drive the vehicle 102. Further, based on the image data and the speech data the application server 106 may determine that the occupant 110 is very diligent and may handle the situation well. Therefore, the application server 106 may determine that the severity level of the in-vehicle emergency may be low and the context may indicate that the occupant 110 is conscious, alone, and is capable of handling the situation.

Based on the detected in-vehicle emergency and the determined severity level and the context of the in-vehicle emergency, the application server 106 selects one or more responses from the plurality of responses. The selected responses may include response actions such as informing nearest traffic controller, unlocking the central lock of the vehicle 102, displaying textual and graphical content (for example, an emergency message such as “CLEAR PATH, BRAKES FAIL”) on the external vehicular display 326, playing an audio message via the audio system of the vehicle 102. The application server 106 may communicate with one or more components of the vehicle 102, and the user device 102a to execute the selected response actions.

Further, the application server 106 may also communicate with a response team (i.e., a traffic police team from a nearest traffic control station) for providing additional emergency assistance. The application server 106 transmits an emergency message describing the emergency and the current location data to the response team that is in vicinity of the current location of the vehicle 102. The response team may track the vehicle 102 based on the current location and visualize real-time conditions inside the vehicle 102 using the image sensor 312 inside the vehicle 102. Further, to provide additional assistance to the occupant 110, the drivers 706a-706c of the near-by vehicles 704a-704c, respectively, and the passer-by individuals 708a-708f, the application server 106 may display textual and graphical content on the external vehicular display 326 to clear way for the vehicle 102. With such effective and efficient way of communication, the various entities (such as the drivers 706a-706c, the passer-by individuals 708a-708f, or the response teams) may give way to the vehicle 102. The application server 106 may then stop the vehicle 102 by shutting off the engine remotely at a safe location.

In an embodiment, the application server 106 is further configured to communicate the emergency message to one or more user device associated with the drivers 706a-706c and the passer-by individuals 708a-708f.

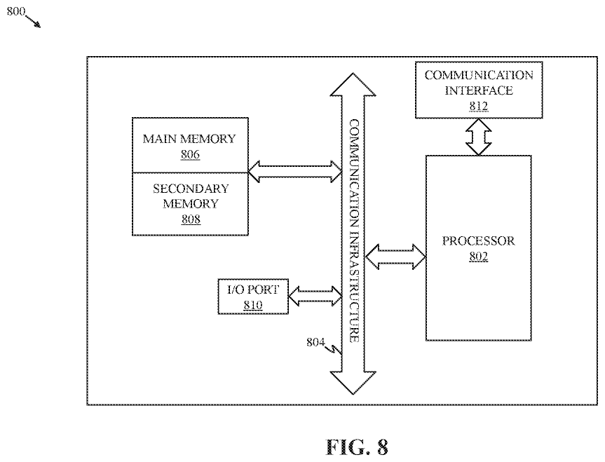

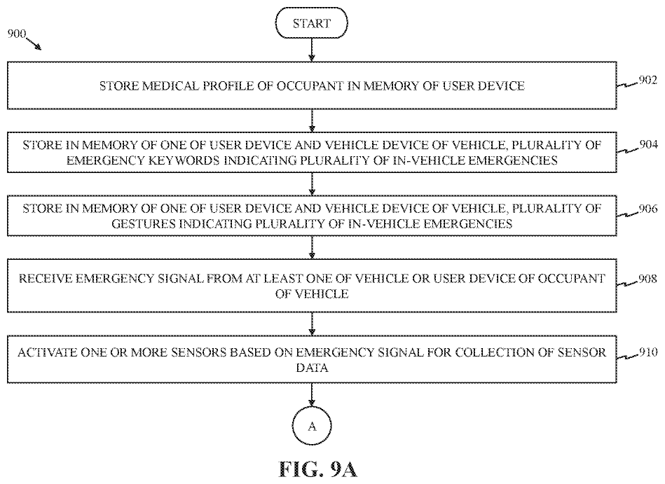

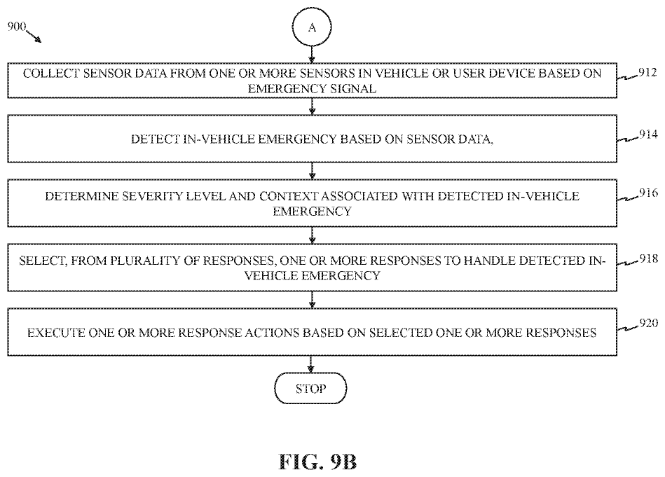

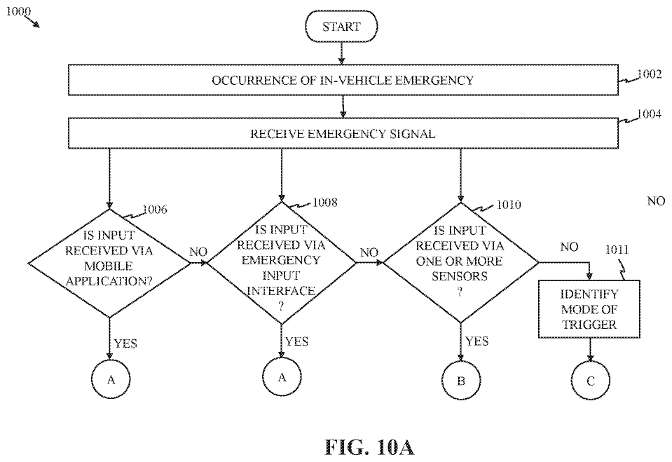

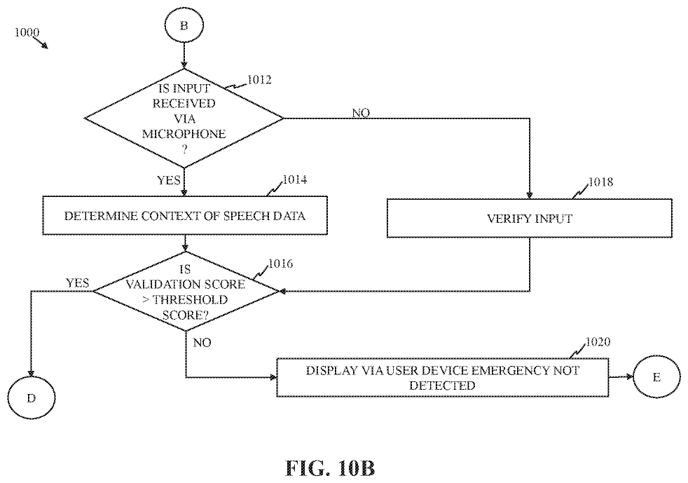

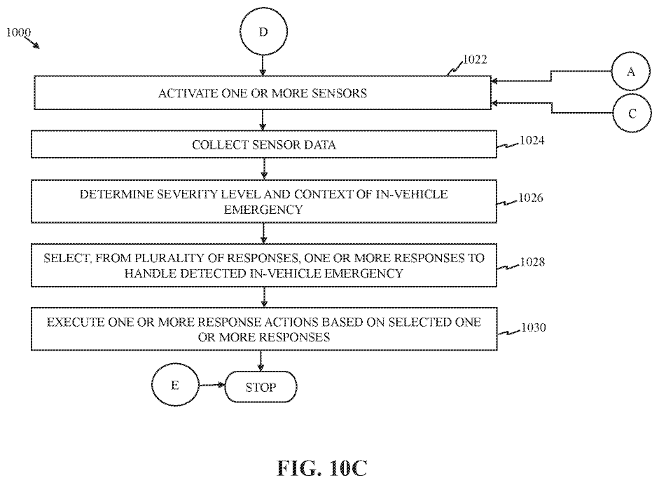

FIG. 8 is a block diagram that illustrates a system architecture of a computer system 800 for in-vehicle emergency detection and response handling, in accordance with an exemplary embodiment of the disclosure. An embodiment of the disclosure, or portions thereof, may be implemented as computer readable code on the computer system 800. In one example, the database server 104 or the application server 106 of FIG. 1 may be implemented in the computer system 800 using hardware, software, firmware, non-transitory computer readable media having instructions stored thereon, or a combination thereof and may be implemented in one or more computer systems or other processing systems. Hardware, software, or any combination thereof may embody modules and components used to implement the methods of FIGS. 9A-9B and 10A-10C.

The computer system 800 may include a processor 802 that may be a special purpose or a general-purpose processing device. The processor 802 may be a single processor or multiple processors. The processor 802 may have one or more processor “cores.” Further, the processor 802 may be coupled to a communication infrastructure 804, such as a bus, a bridge, a message queue, the communication network 108, multi-core message-passing scheme, or the like. The computer system 800 may further include a main memory 806 and a secondary memory 808. Examples of the main memory 806 may include RAM, ROM, and the like. The secondary memory 808 may include a hard disk drive or a removable storage drive (not shown), such as a floppy disk drive, a magnetic tape drive, a compact disc, an optical disk drive, a flash memory, or the like. Further, the removable storage drive may read from and/or write to a removable storage device in a manner known in the art. In an embodiment, the removable storage unit may be a non-transitory computer readable recording media.

The computer system 800 may further include an input/output (I/O) port 810 and a communication interface 812. The I/0 port 810 may include various input and output devices that are configured to communicate with the processor 802. Examples of the input devices may include a keyboard, a mouse, a joystick, a touchscreen, a microphone, and the like. Examples of the output devices may include a display screen, a speaker, headphones, and the like. The communication interface 812 may be configured to allow data to be transferred between the computer system 800 and various devices that are communicatively coupled to the computer system 800. Examples of the communication interface 812 may include a modem, a network interface, i.e., an Ethernet card, a communication port, and the like. Data transferred via the communication interface 812 may be signals, such as electronic, electromagnetic, optical, or other signals as will be apparent to a person skilled in the art. The signals may travel via a communications channel, such as the communication network 108, which may be configured to transmit the signals to the various devices that are communicatively coupled to the computer system 800. Examples of the communication channel may include a wired, wireless, and/or optical medium such as cable, fiber optics, a phone line, a cellular phone link, a radio frequency link, and the like. The main memory 806 and the secondary memory 808 may refer to non-transitory computer readable mediums that may provide data that enables the computer system 800 to implement the methods illustrated in FIGS. 9A-9B and 10A-10C.

FIGS. 9A and 9B, collectively illustrate a flowchart 900 of a method for in-vehicle emergency detection and response handling, in accordance with an exemplary embodiment of the disclosure.

At 902, a medical profile of each of the occupants 110 and 112 is stored in the memory of the user device 102a and/or the passenger device 306. The application server 106 may be configured to store the medical profiles of the occupants 110 and 112 in the memory of the user device 102a and/or the passenger device 306. An emergency signal is generated based on a deviation of the current health of any of the occupants 110 and 112 from the stored medical profile.

At 904, the plurality of emergency keywords indicating the plurality of in-vehicle emergencies are stored locally in memory of the user device 102a, the passenger device 306, and/or the vehicle device of the vehicle 102. The application server 106 may be configured to store, in the memory of the user device 102a, the passenger device 306, and/or the vehicle device of the vehicle 102, the plurality of emergency keywords indicating the plurality of in-vehicle emergencies. An emergency signal is generated by one of the user device 102a, the passenger device 306, and/or the vehicle device of the vehicle 102 based on the utterance of one or more emergency keywords from the plurality of emergency keywords by any of the occupants 110 and 112.

At 906, the plurality of gestures indicating the plurality of in-vehicle emergencies are stored locally in a memory of the user device 102a, the passenger device 306, and/or the vehicle device of the vehicle 102. The application server 106 may be configured to store, in the memory of the user device 102a, the passenger device 306, and/or the vehicle device of the vehicle 102, the plurality of gestures indicating the plurality of in-vehicle emergencies. The emergency signal is generated by any of the user device 102a, the passenger device 306, and/or the vehicle device of the vehicle 102 based on the action of the occupants 110 and 112 that results in one or more gestures of the plurality of gestures indicating the plurality of in-vehicle emergencies.

At 908, the emergency signal is received from the vehicle 102 or a user device (e.g., the user device 102a and the passenger device 306) of any of the occupants 110 and 112 of the vehicle 102. The application server 106 may be configured to receive the emergency signal from the vehicle 102, the user device 102a, or the passenger device 306. The emergency signal may be received based on the input received via one of the plurality of sensors, the mobile application running on the user device 102a or the passenger device 306, and the emergency input interface of the vehicle 102. The application server 106 may be configured to receive the emergency signal based on the deviation of the current health of the occupants 110 and 112 from the stored medical profiles.

At 910, the plurality of sensors are activated based on the emergency signal for collection of the sensor data. The application server 106 may be configured to activate the plurality of sensors based on the emergency signal for the collection of the sensor data. The plurality of sensors include the microphone 310, the image sensor 312, the impact sensor 314, the temperature sensor 316, the smoke sensor 318, the pressure sensor 320, the speed sensor 322, the proximity sensor 324, the GPS sensor 328, or the like (as described in the foregoing description of FIGS. 1 and 3).

At 912, the sensor data is collected from the plurality of sensors in the vehicle 102 or the user device (e.g., the user device 102a or the passenger device 306) based on the emergency signal. The application server 106 may be configured to collect the sensor data from the plurality of sensors in the vehicle 102, the user device 102a, and the passenger device 306 based on the emergency signal.

At 914, in-vehicle emergency is detected based on the sensor data. The application server 106 may be configured to detect the in-vehicle emergency based on the sensor data. The in-vehicle emergency corresponds to at least one of the emergency associated with any of the occupants 110 and 112 and the emergency associated with the vehicle 102.

At 916, the severity level and the context of the detected in-vehicle emergency is determined. The application server 106 may be configured to determine the severity level and the context associated with the detected in-vehicle emergency based on the sensor data.

At 918, one or more responses are selected from the plurality of responses to handle the detected in-vehicle emergency. The application server 106 may be configured to select from the plurality of responses, one or more responses to handle the detected in-vehicle emergency. The one or more responses are selected based on the determined severity level and the determined context of the in-vehicle emergency.

At 920, the one or more response actions based on the selected responses are executed. The application server 106 may be configured to execute the one or more response actions based on the selected responses. For executing the one or more response actions, the application server 106 may be configured to communicate the instruction to at least one of the user device 102a, the emergency contacts of the occupants 110 and 112, the response team associated with the in-vehicle emergency, and one or more user devices associated with one or more users present within the first distance from the vehicle 102 when the in-vehicle emergency is detected.

In one embodiment, for executing the one or more response actions, the application server 106 may be further configured to cause the external vehicular display 326 of the vehicle 102 to display the textual and graphical content indicating the detected in-vehicle emergency. In another embodiment, for executing the one or more response actions, the application server 106 may be further configured to communicate a contextual message or a pre-recorded voice message to the emergency contact of the occupants 110 and/or 112. In another embodiment, for executing the one or more response actions, the application server 106 may be further configured to display an emergency assistance message on the user device 102a of the occupant 110 or the passenger device 306 of the occupant 112. In another embodiment, for executing the one or more response actions, the application server 106 may be further configured to provide navigation assistance to the occupants 110 and 112 of the vehicle 102 via the user device 102a, the passenger device 306, or the vehicle device of the vehicle 102. In another embodiment, for executing the one or more response actions, the application server 106 may be further configured to control remotely the central lock of the vehicle 102 for locking or unlocking. In another embodiment, for executing the one or more response actions, the application server 106 may be further configured to control remotely the driving speed of the vehicle 102 to halt the vehicle 102. In another embodiment, for executing the one or more response actions, the application server 106 may be further configured to initiate a troubleshooting of the vehicle 102.

FIGS. 10A, 10B, and 10C, collectively illustrate an exemplary flowchart 1000 of a method for in-vehicle emergency detection and response handling, in accordance with an exemplary embodiment of the disclosure.

At 1002, an in-vehicle emergency occurs in the vehicle 102. The in-vehicle emergency associated with the vehicle 102 or any of the occupants 110 and 112 of the vehicle 102 may occur. The in-vehicle emergency associated with the vehicle 102 may be an accident of the vehicle 102 or an unlawful incident associated with the vehicle 102. The unlawful incident may be a counterfeit of one or more components of the vehicle 102, a theft of the one or more components, a deliberate damage caused to the vehicle 102, or the like. The in-vehicle emergency associated with any of the occupants 110 and 112 may be a medical emergency, an accident of the vehicle 102, and an unlawful activity. The medical emergency associated with the occupant 110 or 112 may be a health-related constraint that may cause the occupant 110 or 112 to be unfit for further travelling. In one example, the medical emergency associated with the occupant 110 may be an asthma attack, a seizure, a brain stroke, and the like. The accidental emergency may be the accident of the vehicle 102 resulting in physical damage to the occupants 110 and 112 and the unlawful incident emergency may be an assault caused to the occupant 110 or 112, an abduction incident, or the like. Further based on the occurrence of the in-vehicle emergency an emergency signal is generated.

At 1004, the emergency signal is received. The application server 106 may be configured to receive the emergency signal from the user device 102a, the passenger device 306, or the vehicle device of the vehicle 102. The emergency signal may be received based on the generation of the emergency signal via one or more modes of generating (or triggering) emergency signal. The modes of generating the emergency signal may be one of the mobile application hosted on the user device 102a, the mobile application hosted on the passenger device 306, the plurality of sensors installed in the vehicle 102, and/or the emergency input interface of the vehicle 102.

In one embodiment, the emergency signal may be generated based on the input received via the mobile application. In one example, the emergency signal may be generated manually by providing input via the mobile application to initiate the emergency assistance. In such example, the input may be provided based on a health issue, an assault, a burglary, an accident, and the like faced by any of the occupant 110 and 112.

In another embodiment, the emergency signal may be generated based on the input received via the plurality of sensors. In one example, the impact sensor 314 may detect a shock caused to the vehicle 102. Based on the detected shock, the impact sensor 314 may communicate the emergency signal to the application server 106. In one embodiment, the impact sensor 314 may communicate a notification to the mobile application to communicate the emergency signal to the application server 106. In another embodiment, the impact sensor 314 may communicate the notification to the telematics device or the OBD device of the vehicle 102 to communicate the emergency signal to the application server 106. Similarly, the emergency signal may be generated based on the input received via other plurality of sensors.

In another embodiment, the emergency signal may be generated based on the input received via the emergency input interface. In one example, the input may be received via the physical input buttons 308a and 308b based on a potential burglary attempt.

At 1006, determine whether the input is received via the mobile application. The application server 106 may be configured to determine whether the input for generating the emergency signal is received via the mobile application. In other words, the application server 106 determines whether the mode of trigger of the emergency signal is the mobile application being executed on the user device 102a or the passenger device 306. If at 1006, the application server 106 determines that the input is not received via the mobile application, 1008 is executed.

At 1008, determine whether the input is received via the emergency input interface of the vehicle 102. The application server 106 may be configured to determine whether the input is received via the emergency input interface, i.e., any of the physical input buttons 308a and 308b. In other words, the application server 106 determines whether the mode of trigger of the emergency signal is the emergency input interface. If at 1008, the application server 106 determines that the input is not received via the emergency input interface, 1010 is executed.

At 1010, determine whether the emergency signal is received via the plurality of sensors. The application server 106 may be configured to determine whether the input is received via the plurality of sensors. If at 1010, the application server 106 determines that the input is received based on the sensor data detected by one or more of the plurality of sensors, 1012 is executed.

At 1012, determine whether the input is received via the microphone 310 of the vehicle 102. The application server 106 may be configured to determine whether the input is received via the microphone 310. If at 1010, the application server 106 determines that the input is received via the microphone 310, 1014 is executed. At 1014, context of speech data included in the emergency signal is determined. The application server 106 may be configured to determine the context of speech data by analyzing the speech data by using NLP. Determination of the context of the speech data includes determination of the distress level associated with the speech data, a pitch or a repetition of the emergency keywords, a tonality of the speech data, or the like. Upon analyzing the emergency keywords, 1016 is executed. If at 1012, the application server 106 determines that the input is not received via the microphone 310, 1018 is executed.

At 1018, the input is verified. The application server 106 may be configured to verify whether the input is received from other plurality of sensors in the vehicle 102. The application server 106 may be configured to verify the input to determine the mode of trigger of the emergency signal.

At 1014, determine whether a validation score associated with the input is greater than a threshold score. The application server 106 may be configured to determine whether the validation score associated with the input is greater than the threshold score. The threshold score corresponds to a borderline score for detecting false positives and false negatives. The application server 106 may determine whether the validation score associated with the input that resulted in the generation of the emergency signal is above the threshold. If at 1016, the application server 106 determines that the validation score is less than the threshold score, 1020 is executed.

At 1020, an emergency not detected notification is displayed via the user device 102a or the passenger device 306. The application server 106 may be configured to render a user interface, via the user device 102a or the passenger device 306, displaying the emergency not detected notification. Upon displaying the notification, the emergency assistance is terminated. Based on the notification, the emergency assistance may be initiated again by providing a manual input via the rendered user interface for generating the emergency signal. Beneficially, such displaying of the notification eliminates a probability of the false negative during the in-vehicle emergency. In an embodiment, the notification is only displayed when the emergency signal is generated based on manual inputs provided by any of the occupants 110 and 112 such as by pressing the physical emergency buttons 308a and 308b or the touch-based input buttons. If at 1018, the application server 106 determines that the input is above the threshold value, 1022 is executed.

At 1022, the plurality of sensors are activated. The application server 106 may be configured to activate the plurality of sensors to validate the in-vehicle emergency. In one example, the application server 106 may activate the plurality of sensors via one of the user device 102a, the telematics device, and the OBD device.

At 1024, the sensor data is collected. The application server 106 may be configured to collect the sensor data via one of the user device 102a, the telematics device, the OBD device, the passenger device 306, or the like.

At 1026, the severity level and the context of the detected in-vehicle emergency is determined. The application server 106 may be configured to determine the severity level and the context of the in-vehicle emergency. The application server 106 determines the severity level and the context of the in-vehicle emergency based on the sensor data (as described in the foregoing description of FIGS. 1-7).

At 1028, one or more responses from the plurality of responses are selected to handle the in-vehicle emergency. The application server 106 may be configured to select the one or more responses from the plurality of responses to handle the in-vehicle emergency. The application server 106 may be configured to select the one or more responses based on the severity level and the context of the in-vehicle emergency.

At 1030, the one or more response actions based on the selected responses are executed. The application server 106 may be configured to execute the response actions based on the selected responses. The application server 106, for executing the one or more response actions, communicates the instruction to the user device 102a, the emergency contact of the occupant 110 or 112, the response team associated with the in-vehicle emergency, and one or more user devices associated with one or more users present within the first distance from the vehicle 102 when the in-vehicle emergency is detected. The first distance may refer to a vicinity of the vehicle 102 that is a distance which may be travelled shortly to reach the vehicle 102. Examples of the first distance may include 10 meters, 20 meters, 30 meters, 40 meters, and so forth.

The application server 106 may be further configured to execute the one or more response actions by causing the external vehicular display 326 of the vehicle 102 to display the textual and graphical content indicating the detected in-vehicle emergency. The application server 106 may be further configured to execute the one or more response actions by communicating the message or the pre-recorded voice message to the emergency contact of the occupant 110 or 112. The application server 106 may be further configured to execute the one or more response actions by displaying the emergency assistance message on the user device 102a of the occupant 110 or the passenger device 306 of the occupant 112. The application server 106 may be further configured to execute the one or more response actions by controlling remotely the central lock of the vehicle 102 for locking or unlocking. The application server 106 may be further configured to execute the one or more response actions by controlling remotely the driving speed of the vehicle 102 to halt the vehicle 102. The application server 106 may be further configured to execute the one or more response actions by initiating the troubleshooting of the vehicle 102. The application server 106 may be further configured to execute the one or more response actions by activating the audio system, for example, activating a siren on an external speaker of the vehicle 102.

If at 1006, the application server 106 determines that the input is received via the mobile application, 1022 is executed. If at 1008, the application server 106 determines that the input is received via the emergency input interface, 1022 is executed. If at 1010, the application server 106 determines that the input is not received via the plurality of sensors, 1011 is executed. At 1011, a mode of trigger of the emergency signal is identified. The application server 106 may be configured to identify the mode of trigger of the emergency signal based on the data included in the emergency signal and 1022 is executed.

In an embodiment, the application server 106 may be implemented as a local server without departing from scope of the disclosure. The application server 106 may be communicably coupled with the vehicle 102, the user device 102a, the passenger device 306, and the vehicle device.

Various embodiments of the disclosure provide the application server 106 for detecting in-vehicle emergencies and handling emergency responses for the detected in-vehicle emergencies. The application server 106 may be configured to store, the medical profiles of the occupants 110 and 112 in the memory of the user device 102a and the passenger device 306, respectively. The emergency signal may be generated based on the deviation of the current health state of the occupants 110 and 112 from the stored medical profiles. The application server 106 may be further configured to store, in the memory of one of the user device 102a, the passenger device 306, and the vehicle device of the vehicle 102, the plurality of emergency keywords indicating the plurality of in-vehicle emergencies. The emergency signal may be generated based on the utterance of one or more emergency keywords from the plurality of emergency keywords by the occupant. The application server 106 may be further configured to store, in the memory of one of the user device 102a, the passenger device 306, and the vehicle device of the vehicle 102, the plurality of gestures indicating the plurality of in-vehicle emergencies. The emergency signal may be generated based on the action of the occupants 110 and 112 that results in one or more gestures of the plurality of gestures.

The application server 106 may be further configured to receive the emergency signal from one of the vehicle 102, the user device 102a, or the passenger device 306 of the occupants 110 and 112 of the vehicle 102. The emergency signal is received based on the input received via one of the plurality of sensors, the mobile application hosted on the user device 102a and the passenger device 306, and the emergency input interface of the vehicle 102. The application server 106 may be further configured to activate the plurality of sensors based on the emergency signal for the collection of the sensor data. The plurality of sensors include the microphone 310, the image sensor 312, the impact sensor 314, the temperature sensor 316, the smoke sensor 318, the pressure sensor 320, the speed sensor 322, the proximity sensor 324, the global positioning system (GPS) sensor 328, or the like. The application server 106 may be further configured to collect the sensor data from the plurality of sensors in the vehicle 102, the user device 102a, or the passenger device 306 based on the emergency signal. The application server 106 may be further configured to determine the severity level and the context associated with the detected in-vehicle emergency based on the sensor data. The application server 106 may be further configured to select from the plurality of responses, one or more responses to handle the detected in-vehicle emergency. The one or more responses are selected based on the determined severity level and the determined context of the in-vehicle emergency. The application server 106 may be further configured to execute the one or more response actions based on the selected responses. For executing the one or more response actions, the application server 106 may be configured to communicate the instruction to at least one of the user device 102a, the passenger device 306, the emergency contact of the occupants 110 and 112, the response team associated with the in-vehicle emergency, and one or more user devices associated with one or more users present within the first distance from the vehicle 102 when the in-vehicle emergency is detected.

Various embodiments of the disclosure provide a non-transitory computer readable medium having stored thereon, computer executable instructions, which when executed by a computer, cause the computer to execute one or more operations detecting the in-vehicle emergency and handling emergency response for the in-vehicle emergency. The one or more operations include storing the medical profiles of the occupants 110 and 112 in the memory of the user device 102a and the passenger device 306, respectively. The emergency signal may be generated based on the deviation of the current health state of the occupant from the stored medical profile. The one or more operations further include storing in the memory of one of one of the user device 102a, the passenger device 306, and the vehicle device of the vehicle 102, the plurality of emergency keywords indicating the plurality of in-vehicle emergencies. The emergency signal may be generated based on the utterance of one or more emergency keywords from the plurality of emergency keywords by the occupant. The one or more operations further include storing in the memory of one of one of the user device 102a, the passenger device 306, and the vehicle device of the vehicle 102, the plurality of gestures indicating the plurality of in-vehicle emergencies. The emergency signal may be generated based on the action of the occupants 110 and 112 that results in one or more gestures of the plurality of gestures. The one or more operations further include receiving the emergency signal from the vehicle 102, the user device 102a, or the passenger device 306 of the occupants 110 and 112, respectively of the vehicle 102. The emergency signal is received based on the input received via one of the plurality of sensors, the mobile application hosted on the user device 102a, and the emergency input interface of the vehicle 102. The one or more operations further include activating the plurality of sensors based on the emergency signal for the collection of the sensor data. The plurality of sensors the microphone 310, the image sensor 312, the impact sensor 314, the temperature sensor 316, the smoke sensor 318, the pressure sensor 320, the speed sensor 322, the proximity sensor 324, the global positioning system (GPS) sensor 328, or the like. The one or more operations further include collecting the sensor data from the plurality of sensors in the vehicle 102, the user device 102a, the passenger device 306 based on the emergency signal. The one or more operations further include detecting the in-vehicle emergency based on the sensor data. The in-vehicle emergency corresponds to the emergency associated with the occupant and the emergency associated with the vehicle 102. The one or more operations further include determining the severity level and the context associated with the detected in-vehicle emergency based on the sensor data. The one or more operations further include selecting, from the plurality of responses, one or more responses to handle the detected in-vehicle emergency. The one or more responses are selected based on the determined severity level and the determined context. The one or more operations further include executing the one or more response actions based on the selected one or more responses. The step of executing the one or more response actions include communicating the instruction to the user device102a, the passenger device 306, the emergency contact of the occupants 110 and 112, the response team associated with the in-vehicle emergency, and the one or more user devices associated with one or more users present within the first distance from the vehicle 102 when the in-vehicle emergency is detected.

The disclosed embodiments encompass numerous advantages. Exemplary advantages of the disclosed methods include, but are not limited to, ensuring a round the clock emergency assistance to handle any type of in-vehicle emergency associated with the vehicle 102 and/or the occupants 110 and 112. The disclosed methods and systems ensure an efficient and optimized detection of the in-vehicle emergencies. The disclosed methods and systems allow for generation of the emergency signal manually by the occupants 110 and 112 of the vehicle 102 or automatically based on the sensor data collected by the plurality of sensors. Therefore, the disclosed methods and systems ensure that the in-vehicle emergency is always detected. Further, the disclosed methods and systems ensure that the in-vehicle emergency is handled based on the severity level and the context of the in-vehicle emergency. The emergency response handling based on the severity level and the context of the in-vehicle emergency allows for providing suitable emergency responses in different scenarios. Therefore, the disclosed methods and systems allow for an efficient and optimal handling of the detected in-vehicle emergency. Moreover, the disclosed methods and systems allow for an elimination of false positive incidents and a detection of false negative incidents. Therefore, the disclosed methods and systems ensure the emergency assistance at all times. The disclosed methods and systems further allow for emergency assistance even when there is no network connectivity. Therefore, the disclosed systems and methods eliminate a necessity of the network availability for providing the emergency assistance. Further, the disclosed methods and systems reduce human intervention, cost, and time required for handling the in-vehicle emergency.

A person of ordinary skill in the art will appreciate that embodiments and exemplary scenarios of the disclosed subject matter may be practiced with various computer system configurations, including multi-core multiprocessor systems, minicomputers, mainframe computers, computers linked or clustered with distributed functions, as well as pervasive or miniature computers that may be embedded into virtually any device. Further, the operations may be described as a sequential process, however some of the operations may in fact be performed in parallel, concurrently, and/or in a distributed environment, and with program code stored locally or remotely for access by single or multiprocessor machines. In addition, in some embodiments, the order of operations may be rearranged without departing from the spirit of the disclosed subject matter.

Techniques consistent with the disclosure provide, among other features, systems and methods for in-vehicle emergency detection and response handling. While various exemplary embodiments of the disclosed systems and methods have been described above, it should be understood that they have been presented for purposes of example only, and not limitations. It is not exhaustive and does not limit the disclosure to the precise form disclosed. Modifications and variations are possible in light of the above teachings or may be acquired from practicing of the disclosure, without departing from the breadth or scope.

While various embodiments of the disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

What is claimed is:

1. A method for emergency response handling, the method comprising:
receiving, by an application server, an emergency signal from at least one of a vehicle or a user device of an occupant of the vehicle;
collecting, by the application server, sensor data from one or more sensors in the vehicle or the user device based on the emergency signal;
detecting, by the application server, an in-vehicle emergency based on the sensor data, wherein the in-vehicle emergency corresponds to at least one of an emergency associated with the occupant and an emergency associated with the vehicle;
determining, by the application server, a severity level and a context associated with the detected in-vehicle emergency based on the sensor data;
selecting, by the application server, from a plurality of responses, one or more responses to handle the detected in-vehicle emergency, wherein the one or more responses are selected based on the determined severity level and the determined context; and
executing, by the application server, one or more response actions based on the selected one or more responses, wherein executing the one or more response actions comprises:
communicating, by the application server, an instruction to at least one of the user device, an emergency contact of the occupant, a response team associated with the in-vehicle emergency, and one or more user devices associated with one or more users present within a first distance from the vehicle when the in-vehicle emergency is detected.

2. The method of claim 1, wherein the emergency signal is received based on an input received via one of the one or more sensors, a mobile application hosted on the user device, and an emergency input interface of the vehicle.

3. The method of claim 1, further comprising storing, by the application server, a medical profile of the occupant in a memory of the user device, wherein the emergency signal is received based on a deviation of a current health state of the occupant from the stored medical profile.

4. The method of claim 1, wherein the one or more sensors include at least one of a global positioning system (GPS) sensor, an impact sensor, a temperature sensor, a speed sensor, a smoke sensor, a proximity sensor, a pressure sensor, an image sensor, and a microphone.

5. The method of claim 1, wherein executing the one or more response actions further comprises at least one of:
causing, by the application server, an external vehicular display of the vehicle to display textual and graphical content indicating the detected in-vehicle emergency;
communicating, by the application server, a message or a pre-recorded voice message to the emergency contact of the occupant; and
displaying, by the application server, an emergency assistance message on the user device of the occupant.

6. The method of claim 1, wherein executing the one or more response actions further comprises at least one of:
providing, by the application server, a navigation assistance to the occupant of the vehicle via the user device or a vehicle device of the vehicle;
controlling remotely, by the application server, a central lock of the vehicle for locking or unlocking;
controlling remotely, by the application server, a driving speed of the vehicle to halt the vehicle; and
initiating, by the application server, a troubleshooting of the vehicle.

7. The method of claim 1, further comprising activating, by the application server, the one or more sensors based on the emergency signal for the collection of the sensor data.

8. The method of claim 1, further comprising storing, by the application server, in a memory of one of the user device and a vehicle device of the vehicle, a plurality of emergency keywords indicating a plurality of in-vehicle emergencies, and wherein the emergency signal is received based on an utterance of one or more emergency keywords from the plurality of emergency keywords by the occupant.

9. The method of claim 1, further comprising storing, by the application server, in a memory of one of the user device and a vehicle device of the vehicle, a plurality of gestures indicating a plurality of in-vehicle emergencies, and wherein the emergency signal is received based on an action of the occupant that results in one or more gestures of the plurality of gestures.

10. The method of claim 1, wherein the sensor data includes at least one of image data, speech data of the occupant, temperature data, impact magnitude data, pressure magnitude data, speedometer data, location data, and current health state data, and wherein the current health state data includes blood pressure magnitude data of the occupant, pulse-rate data of the occupant, oxygen level data of the occupant, and state of consciousness of the occupant.

11. A system for emergency response handling, the system comprising:
an application server configured to:
receive, an emergency signal from at least one of a vehicle or a user device of an occupant of the vehicle;
collect, sensor data from one or more sensors in the vehicle or the user device based on the emergency signal;
detect, an in-vehicle emergency based on the sensor data, wherein the in-vehicle emergency corresponds to at least one of an emergency associated with the occupant and an emergency associated with the vehicle;
determine, a severity level and a context associated with the detected in-vehicle emergency based on the sensor data;
select, from a plurality of responses, one or more responses to handle the detected in-vehicle emergency, wherein the one or more responses are selected based on the determined severity level and the determined context; and
execute, one or more response actions based on the selected one or more responses, wherein a first response action of the one or more response actions includes communication of an instruction to at least one of the user device, an emergency contact of the occupant, a response team associated with the in-vehicle emergency, and one or more user devices associated with one or more users present within a first distance from the vehicle when the in-vehicle emergency is detected.

12. The system of claim 11, wherein the emergency signal is received based on an input received via one of the one or more sensors, a mobile application hosted on the user device, and an emergency input interface of the vehicle.

13. The system of claim 11, wherein the application server is further configured to store a medical profile of the occupant in a memory of the user device, wherein the emergency signal is received based on a deviation of a current health state of the occupant from the stored medical profile.

14. The system of claim 11, wherein the one or more sensors include at least one of a global positioning system (GPS) sensor, an impact sensor, a temperature sensor, a speed sensor, a smoke sensor, a proximity sensor, a pressure sensor, an image sensor, and a microphone.

15. The system of claim 11, wherein to execute the one or more response actions, the application server is further configured to cause an external vehicular display of the vehicle to display a predefined text indicating the detected in-vehicle emergency.

16. The system of claim 11, wherein to execute the one or more response actions, the application server is further configured to provide a navigation assistance to the occupant of the vehicle.

17. The system of claim 11, wherein to execute the one or more response actions, the application server is further configured to:
remotely control a central lock of the vehicle to lock or unlock; and
remotely control a driving speed of the vehicle to halt the vehicle.

18. The system of claim 11, wherein the application server is further configured to activate the one or more sensors based on the emergency signal for the collection of the sensor data.

19. The system of claim 11, wherein the application server is further configured to store, in a memory of one of the user device and a vehicle device of the vehicle, a plurality of emergency keywords that indicate a plurality of in-vehicle emergencies, and wherein the emergency signal is received based on an utterance of one or more emergency keywords from the plurality of emergency keywords by the occupant.

20. The system of claim 11, wherein the application server is further configured to store, in a memory of one of the user device and a vehicle device of the vehicle, a plurality of gestures that indicate a plurality of in-vehicle emergencies, and wherein the emergency signal is received based on an action of the occupant that results in one or more gestures of the plurality of gestures.
